(19) United States
(12) Patent Application Publication
(10) Pub. No.: US 2026/0367579 A1
(43) Pub. Date: Nov. 19, 2026

(54) MECHANICAL WATCH WITH REMOVABLE SENSOR ASSEMBLY

(71) Applicant: Masimo Corporation, Irvine, CA (US)

(72) Inventor: Stephen Scruggs, Newport Beach, CA (US)

(21) Appl. No.: 19/673,863

(22) Filed: May 11, 2026

Related U.S. Application Data
(60) Provisional application No. 63/805,022, filed on May 13, 2025.

(51) Int. Cl. G04B 47/06; A61B 5/00; A61B 5/01; G01D 11/24; G01D 11/30; G01K 13/20; G04B 37/00; H05K 5/02

(52) U.S. Cl. CPC G04B 47/063; A61B 5/0059; A61B 5/01; A61B 5/681; G01D 11/245; G01D 11/30; G01K 13/20; G04B 37/0008; G04B 47/066; G04B 47/068; H05K 5/0243; A61B 2560/0214; A61B 2562/0233; A61B 2562/0271; A61B 2562/166

(57) ABSTRACT

An electronic sensor assembly can removably mechanically connect with a mechanically powered watch. The sensor assembly can have a circuit board, physiological sensors, such as an oximeter, which can be mounted to the circuit board, one or more hardware processors for processing data from the physiological sensors, a battery for powering components of the sensor assembly, and a communication component that can communicate with remote computing devices. The sensor assembly can be electrically isolated from the mechanically powered watch which may not have electrical components. The sensor assembly can operate independent of the mechanically powered watch and can continue to operate whether mechanically connected to, or disconnected from, the mechanically powered watch.

### CROSS REFERENCE TO RELATED APPLICATIONS

This application claims priority to U.S. Provisional Patent Application No. 63/805,022, filed May 13, 2025, which is incorporated herein by reference in its entirety. Any and all applications for which a foreign or domestic priority claim is identified in the Application Data Sheet as filed with the present application are hereby incorporated by reference under 37 CFR 1.57.

### TECHNICAL FIELD

The present disclosure relates to a wearable device, such as a mechanical watch, with a removable sensor assembly for measuring and/or monitoring a user's physiological information.

### BACKGROUND

Mechanical watches are generally powered by mechanical power sources such as one or more springs that can store energy. Mechanical watches generally include mechanical timekeeping components such as one or more gears, a balance wheel, and/or an escapement to track the passage of time. Traditional mechanical watches typically lack electronic components such as a battery, a digital display, and/or one or more sensors to measure and/or monitor a user's physiological information.

### SUMMARY

Smart watches and/or digital watches with sensors that measure and/or monitor a user's physiological information are increasingly popular. However, some individuals prefer mechanical watches to smart watches and/or digital watches. Traditional digital watches and/or smart watches can have relatively short life spans compared to mechanical watches due to their dependency on electronic components such as digital displays and/or batteries. Digital watches and/or smart watches are frequently replaced with newer models, while mechanical watches are often maintained for many years as family heirlooms. Digital watches and/or smart watches must be periodically charged to function, while traditional mechanical watches do not require charging. Some individuals prefer the look and feel of mechanical watches over digital watches and/or smart watches.

Traditional mechanical watches lack the ability to measure and/or monitor a user's physiological information, a feature that is increasingly desirable to many watch users. Therefore, there exists a need for a mechanical watch that maintains the perceived benefits of traditional mechanical watches while being capable of measuring and/or monitoring a user's physiological information. Accordingly, the present disclosure provides a mechanical watch with a removable sensor assembly for measuring and/or monitoring a user's physiological information. Such a device can advantageously provide individuals with many of the advantages of a traditional mechanical watch while simultaneously allowing a user to measure and/or monitor his or her physiological information.

The present disclosure relates in some aspects to a mechanical watch with a removable sensor assembly that can removably mechanically connect with the mechanical watch. The mechanical watch can be mechanically powered and/or include no electronic components. The sensor assembly can connect to the mechanical watch such that the sensor assembly is held against the tissue (e.g., skin) of a user when the sensor assembly is connected to the mechanical watch and the mechanical watch is worn by the user. The sensor assembly can be connected to the mechanical watch by rotating the sensor assembly in a first direction and/or disconnected from the mechanical watch by rotating the sensor assembly in a second direction. The sensor assembly can have a circuit board, physiological sensors, such as an oximeter, which can be mounted to the circuit board, one or more hardware processors for processing data from the physiological sensors, a battery for powering components of the sensor assembly, and/or a communication component that can communicate with remote computing devices. The sensor assembly can be electrically isolated from the mechanical watch. The sensor assembly can operate independently of the mechanically powered watch such that the sensor assembly can continue to operate when it is mechanically disconnected from the mechanical watch.

In some variants, a sensor assembly is disclosed herein. The sensor assembly can include a housing that can removably mechanically connect with a mechanically powered watch. The housing can include a curved surface that can contact tissue of a user when the sensor assembly is connected with the mechanically powered watch and the mechanically powered watch is worn by the user. The sensor assembly can include a circuit board housed by the housing. The sensor assembly can include a plurality of emitters mounted to the circuit board that can emit optical radiation through one or more windows of the housing toward tissue of the user. The sensor assembly can include a plurality of detectors mounted to the circuit board. The plurality of detectors can output one or more signals responsive to detecting optical radiation emitted from the plurality of emitters after attenuation by the tissue. The sensor assembly can include one or more hardware processors configured to process the one or more signals from the plurality of detectors to generate processed physiological data. The sensor assembly can include a communication component that can communicate the processed physiological data to a remote computing device. The sensor assembly can include a battery housed by the housing that can provide power to the circuit board, the one or more hardware processors, and the communication component. The sensor assembly can include one or more charging ports embedded within the curved surface that houses one or more charging pins. The one or more charging pins can be electrically connected with the battery. In some variants, the sensor assembly is not electrically connected with the mechanically powered watch and can operate independently of the mechanically powered watch. In some variants, the sensor assembly is electrically isolated from the mechanically powered watch.

In some variants, the sensor assembly can include a shroud forming at least a portion of the curved surface. In some variants, the shroud surrounds the one or more windows. In some variants, the shroud is removably connected with the housing. In some variants, the shroud has a different color and/or texture than the housing.

In some variants, the sensor assembly can include a temperature sensor mounted to the circuit board that can respond to thermal energy from the user.

In some variants, the housing can include one or more ribs that can slide within the mechanically powered watch to secure the sensor assembly to the mechanically powered watch. In some variants, the sensor assembly can rotate in a first direction relative to the mechanically powered watch to mechanically connect the housing to the mechanically powered watch and rotate in a second direction relative to the mechanically powered watch to mechanically disconnect the housing from the mechanically powered watch. In some variants, the housing can include a surface configured to contact the mechanically powered watch to inhibit the sensor assembly from over rotating in the first direction.

In some variants, at least a portion of the sensor assembly is circular.

In some variants, a mechanically powered watch is disclosed herein. The mechanically powered watch can include a face disposed on a front portion of the mechanically powered watch that can indicate time, one or more gears that can control the face, a spring that can provide power to the timekeeping components, and/or a receptacle on a back portion of the mechanically powered watch opposite the front portion. The receptacle can removably mechanically connect with an electronic sensor assembly. The receptacle can hold the electronic sensor assembly against tissue of a user when the mechanically powered watch is worn by the user.

In some variants, the mechanically powered watch can include a rail that can mechanically mate with the sensor assembly to hold the sensor assembly to the receptacle. In some variants, the mechanically powered watch can include a barrier that can inhibit the electronic sensor assembly from rotating beyond a desired orientation relative to the receptacle. In some variants, the receptacle can removably mechanically connect with a cover when the electronic sensor assembly is mechanically disconnected from the receptacle. In some variants, the timekeeping components operate independently from the electronic sensor assembly and continue to operate when the electronic sensor assembly is mechanically disconnected from the receptacle.

In some variants, the mechanically powered watch does not have an electronic display. In some variants, the mechanically powered watch does not have a battery.

In some variants, a wearable device is disclosed herein. The wearable device can include a mechanically powered watch. The mechanically powered watch can include a face disposed on a front portion of the mechanically powered watch that can indicate time, timekeeping components including one or more gears that can control the face, a spring that can provide power to the timekeeping components, and/or a receptacle on a back portion of the mechanically powered watch opposite the front portion. The wearable device can include a sensor assembly. The wearable device can include a housing that can removably mechanically connect with the receptacle of the mechanically powered watch such that the sensor assembly is held against the mechanically powered watch. The housing can include a curved surface that can contact tissue of a user when the sensor assembly is connected with the receptacle and the wearable device is worn by the user. The sensor assembly can include a circuit board housed by the housing. The sensor assembly can include a plurality of emitters mounted to the circuit board that can emit optical radiation through one or more windows of the housing toward tissue of the user. The sensor assembly can include a plurality of detectors mounted to the circuit board that can output one or more signals responsive to detecting optical radiation emitted from the plurality of emitters after attenuation by said tissue. The sensor assembly can include one or more hardware processors that can process the one or more signals from the plurality of detectors to generate processed physiological data. The sensor assembly can include a communication component that can communicate the processed physiological data to a remote computing device. The sensor assembly can include a battery housed by the housing that can provide power to the circuit board, the one or more hardware processors, and the communication component. The sensor assembly can include one or more charging ports embedded within the curved surface and housing one or more charging pins. The one or more charging pins can be electrically connected with the battery. In some variants, the sensor assembly is not electrically connected with the mechanically powered watch and is can operate independently of the mechanically powered watch. The receptacle can hold the sensor assembly against tissue of the user when the sensor assembly is connected with the receptacle and the wearable device is worn by the user.

In some variants, the sensor assembly can rotate in a first direction relative to the receptacle to mechanically connect the housing to the receptacle and rotate in a second direction relative to the receptacle to mechanically disconnect the housing from the receptacle. In some variants, the housing can include one or more ribs that can slide within the receptacle to secure the sensor assembly to the receptacle. In some variants, the mechanically powered watch can include a rail within the receptacle that can mechanically mate with the one or more ribs when the one or more ribs slide within the receptacle to secure the sensor assembly to the receptacle.

In some variants, the housing can include a surface configured to contact the mechanically powered watch to inhibit the sensor assembly from rotating in the first direction beyond a desired orientation relative to the receptacle. In some variants, the mechanically powered watch can include a barrier extending into the receptacle that can contact the surface of the housing to inhibit the sensor assembly from rotating in the first direction beyond a desired orientation relative to the receptacle.

In some variants, the sensor assembly can include a shroud that can removably connect to the sensor assembly. The shroud can surround the one or more windows when connected to the sensor assembly.

In some variants, the sensor assembly is electrically isolated from the mechanically powered watch. In some variants, the timekeeping components operate independently from the sensor assembly and continue to operate when the sensor assembly is mechanically disconnected from the receptacle.

In some variants, the receptacle can removably mechanically connect with a cover when the sensor assembly is mechanically disconnected from the receptacle.

In some variants, a sensor assembly is disclosed herein. The sensor assembly can include a housing including one or more ribs configured to slide within a mechanically powered watch to removably mechanically connect the sensor assembly with the mechanically powered watch. The housing can include a surface configured to contact tissue of a user when the sensor assembly is connected with the mechanically powered watch and the mechanically powered watch is worn by the user. The sensor assembly can include a circuit board housed by the housing. The sensor assembly can include a plurality of emitters mounted to the circuit board and configured to emit optical radiation through one or more windows of the housing toward tissue of the user. The sensor assembly can include a plurality of detectors mounted to the circuit board, said plurality of detectors configured to output one or more signals responsive to detecting optical radiation emitted from the plurality of emitters after attenuation by said tissue. The sensor assembly can include one or more hardware processors configured to process the one or more signals from the plurality of detectors to generate processed physiological data. The sensor assembly can include a communication component configured to communicate the processed physiological data to a remote computing device. The sensor assembly can include one or more batteries housed by the housing and configured to provide power to the circuit board, the one or more hardware processors, and the communication component. The sensor assembly may not be electrically connected with the mechanically powered watch and may operate independently of the mechanically powered watch.

In some variants, the sensor assembly can include one or more charging ports embedded within the surface and housing one or more charging pins, wherein said one or more charging pins are electrically connected with the battery

In some variants, the sensor assembly is electrically isolated from the mechanically powered watch.

In some variants, the sensor assembly includes a shroud removably coupled with the housing, the shroud forming at least a portion of the surface when coupled with the housing.

In some variants, the shroud surrounds the one or more windows.

In some variants, the shroud has a different color and/or texture than the housing.

In some variants, the sensor assembly can include a temperature sensor mounted to the circuit board and configured to respond to thermal energy from the user.

In some variants, the housing is configured to: rotate in a first direction relative to the mechanically powered watch to mechanically connect the housing to the mechanically powered watch; and rotate in a second direction relative to the mechanically powered watch to mechanically disconnect the housing from the mechanically powered watch.

In some variants, the housing includes a surface configured to contact a wall of the mechanically powered watch to inhibit the sensor assembly from over rotating in the first direction.

In some variants, at least a portion of the sensor assembly is circular.

In some variants, a mechanically powered watch is disclosed herein. The mechanically powered watch can include a face disposed on a front portion of the mechanically powered watch, said face configured to indicate time. The mechanically powered watch can include timekeeping components including one or more gears configured to control the time indicated by the face. The mechanically powered watch can include a spring configured to provide power to the timekeeping components. The mechanically powered watch can include a receptacle on a back portion of the mechanically powered watch opposite the front portion, the receptacle configured to removably mechanically connect with an electronic sensor assembly, the receptacle configured to hold the electronic sensor assembly against tissue of a user when the mechanically powered watch is worn by the user.

In some variants, the mechanically powered watch can include a rail configured to mechanically mate with the sensor assembly to hold the sensor assembly to the receptacle.

In some variants, the mechanically powered watch is configured to: rotate in a first direction relative to the sensor assembly to mechanically connect the sensor assembly with the receptacle; and rotate in a second direction relative to the sensor assembly to mechanically disconnect the sensor assembly from the receptacle.

In some variants, the mechanically powered watch can include a barrier configured to inhibit the sensor assembly from rotating in the first direction beyond a desired orientation relative to the receptacle.

In some variants, the receptacle is configured to removably mechanically connect with a cover when the electronic sensor assembly is mechanically disconnected from the receptacle.

In some variants, the timekeeping components operate independently from the electronic sensor assembly and continue to operate when the electronic sensor assembly is mechanically disconnected from the receptacle.

In some variants, the mechanically powered watch does not have an electronic display.

In some variants, the mechanically powered watch does not have a battery.

In some variants, a wearable device is disclosed herein. The wearable device can include a mechanically powered watch including a face disposed on a front portion of the mechanically powered watch, said face configured to indicate time. The mechanically powered watch can include timekeeping components including one or more gears configured to control the time indicated by the face. The mechanically powered watch can include a spring configured to provide power to the timekeeping components. The mechanically powered watch can include a receptacle on a back portion of the mechanically powered watch opposite the front portion. The wearable device can include a sensor assembly including a housing configured to removably mechanically connect with the receptacle of the mechanically powered watch such that the sensor assembly is held against the mechanically powered watch, said housing having a curved surface configured to contact tissue of a user when the sensor assembly is connected with the receptacle and the wearable device is worn by the user. The sensor assembly can include a circuit board housed by the housing. The sensor assembly can include a plurality of emitters mounted to the circuit board and configured to emit optical radiation through one or more windows of the housing toward tissue of the user. The sensor assembly can include a plurality of detectors mounted to the circuit board, said plurality of detectors configured to output one or more signals responsive to detecting optical radiation emitted from the plurality of emitters after attenuation by said tissue. The sensor assembly can include one or more hardware processors configured to process the one or more signals from the plurality of detectors to generate processed physiological data. The sensor assembly can include a communication component configured to communicate the processed physiological data to a remote computing device; one or more batteries housed by the housing and configured to provide power to the circuit board, the one or more hardware processors, and the communication component. The sensor assembly can include one or more charging ports embedded within the curved surface and housing one or more charging pins, wherein said one or more charging pins are electrically connected with the battery. The sensor assembly may not be electrically connected with the mechanically powered watch and may operate independently of the mechanically powered watch. The receptacle can hold the sensor assembly against tissue of the user when the sensor assembly is connected with the receptacle and the wearable device is worn by the user.

In some variants, the sensor assembly is configured to: rotate in a first direction relative to the receptacle to mechanically connect the housing to the receptacle; and rotate in a second direction relative to the receptacle to mechanically disconnect the housing from the receptacle.

In some variants, the housing further includes one or more ribs configured to slide within the receptacle to secure the sensor assembly to the receptacle.

In some variants, the mechanically powered watch includes a rail within the receptacle, the rail configured to mechanically mate with the one or more ribs when the one or more ribs slide within the receptacle to secure the sensor assembly to the receptacle.

In some variants, the housing further includes a surface configured to contact the mechanically powered watch to inhibit the sensor assembly from rotating in the first direction beyond a desired orientation relative to the receptacle.

In some variants, the mechanically powered watch further includes a barrier extending into the receptacle, said barrier configured to contact the surface of the housing to inhibit the sensor assembly from rotating in the first direction beyond a desired orientation relative to the receptacle.

In some variants, the sensor assembly includes a shroud configured to removably connect to the sensor assembly, the shroud configured to surround the one or more windows when connected to the sensor assembly.

In some variants, the sensor assembly is electrically isolated from the mechanically powered watch.

In some variants, the timekeeping components operate independently from the sensor assembly and continue to operate when the sensor assembly is mechanically disconnected from the receptacle.

In some variants, the receptacle is configured to removably mechanically connect with a cover when the sensor assembly is mechanically disconnected from the receptacle.

Various combinations of the above and below recited features, embodiments, implementations, and aspects are also disclosed and contemplated by the present disclosure.

Additional implementations of the disclosure are described below in reference to the appended claims, which may serve as an additional summary of the disclosure.

In various implementations, systems and/or computer systems are disclosed that comprise a computer-readable storage medium having program instructions embodied therewith, and one or more processors configured to execute the program instructions to cause the systems and/or computer systems to perform operations comprising one or more aspects of the above-and/or below-described implementations (including one or more aspects of the appended claims).

In various implementations, computer-implemented methods are disclosed in which, by one or more processors executing program instructions, one or more aspects of the above-and/or below-described implementations (including one or more aspects of the appended claims) are implemented and/or performed.

In various implementations, computer program products comprising a computer-readable storage medium are disclosed, wherein the computer-readable storage medium has program instructions embodied therewith, the program instructions executable by one or more processors to cause the one or more processors to perform operations comprising one or more aspects of the above-and/or below-described implementations (including one or more aspects of the appended claims).

### BRIEF DESCRIPTION OF THE DRAWINGS

Various implementations will be described hereinafter with reference to the accompanying drawings. These implementations are illustrated and described by example only, and are not intended to limit the scope of the disclosure.

FIGS. 1A-1E illustrate various views of a wearable device with a sensor assembly in accordance with aspects of this disclosure.

FIGS. 2A-2D illustrate various views of the wearable device of FIG. 1A configured to receive a sensor assembly in accordance with aspects of this disclosure.

FIG. 3 illustrates a perspective view of the wearable device of FIG. 1A with an exploded view of the sensor assembly of FIG. 1A in accordance with aspects of this disclosure.

FIGS. 4A-4B illustrate exploded views of a portion of the sensor assembly of FIG. 1A in accordance with aspects of this disclosure.

FIG. 4C illustrates a perspective view of a shroud of the sensor assembly of FIG. 1A in accordance with aspects of this disclosure.

FIGS. 5A-5C illustrate exploded views of the sensor assembly of FIG. 1A in accordance with aspects of this disclosure.

FIGS. 6A-7D illustrate various views of the sensor assembly of FIG. 1A in accordance with aspects of this disclosure.

FIGS. 8A-8D illustrate various views of the sensor assembly of FIG. 1A being installed on the wearable device of FIG. 1A in accordance with aspects of this disclosure.

FIGS. 9A-9F illustrate various views of a back housing of the sensor assembly of FIG. 1A being installed on the wearable device of FIG. 1A in accordance with aspects of this disclosure.

FIG. 10 illustrates a cross-sectional view of the wearable device with a sensor assembly of FIG. 1A in accordance with aspects of this disclosure.

FIG. 11 illustrates a portion of a circuit board of the sensor assembly of FIG. 1A in accordance with aspects of this disclosure.

FIG. 12 illustrates a cross-sectional view of a portion of the sensor assembly of FIG. 1A in accordance with aspects of this disclosure.

FIG. 13 is a block diagram illustrating a sensor assembly in communication with a server and/or a user device over a network.

### DETAILED DESCRIPTION

The present disclosure will now be described with reference to the accompanying figures, wherein like numerals may refer to like elements throughout. The following description is merely illustrative in nature and is in no way intended to limit the disclosure, its application, or uses. Furthermore, the devices, systems, and/or methods disclosed herein can include several novel features, no single one of which is solely responsible for its desirable attributes or which is essential to practicing the devices, systems, and/or methods disclosed herein. Additionally, the structures, systems, and/or devices described herein may be embodied as integrated components or as separate components.

Some aspects and/or implementations have been described in connection with the accompanying drawings. The figures may be drawn to scale, but such scale is not limiting, since dimensions and proportions other than what are shown are contemplated and are within the scope of the disclosed invention. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, any methods described herein may be practiced using any device suitable for performing the recited steps. Various steps within a method may be executed in different order without altering the principles of the present disclosure.

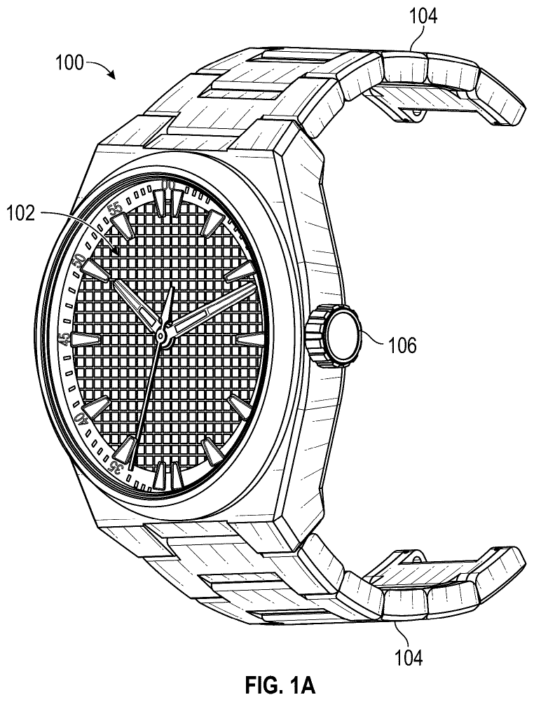

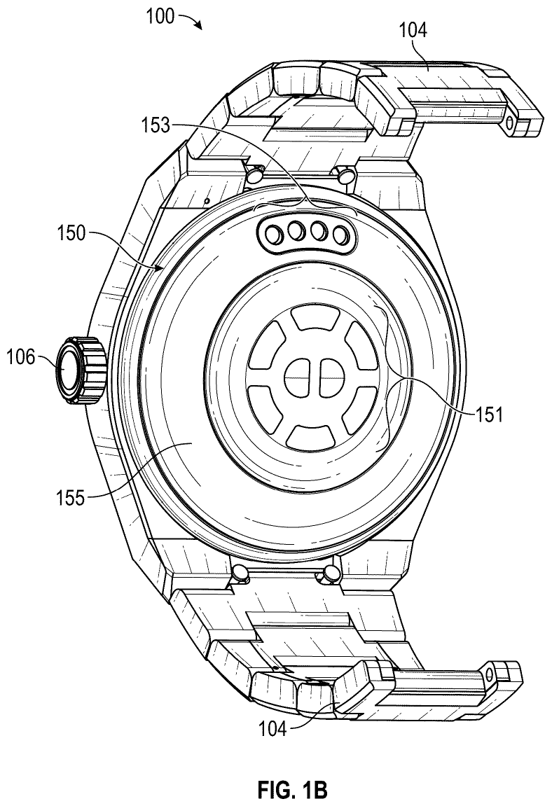

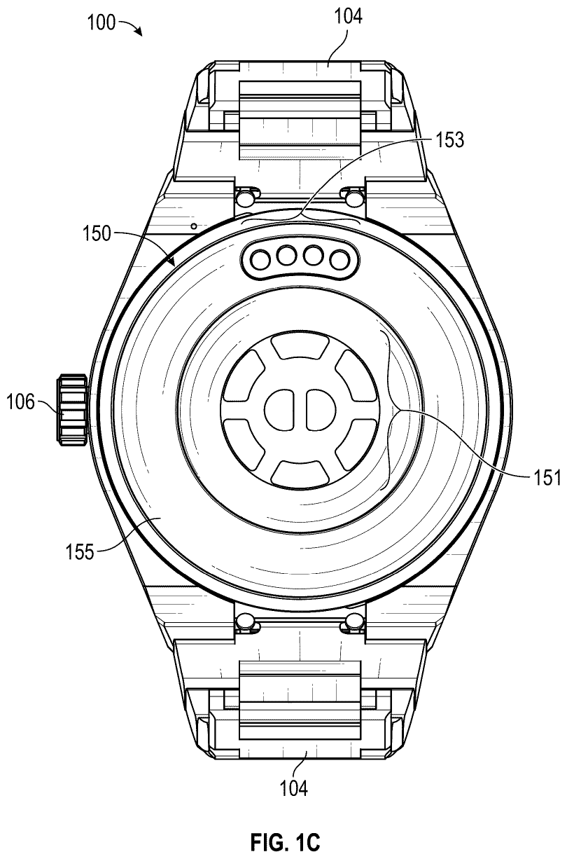

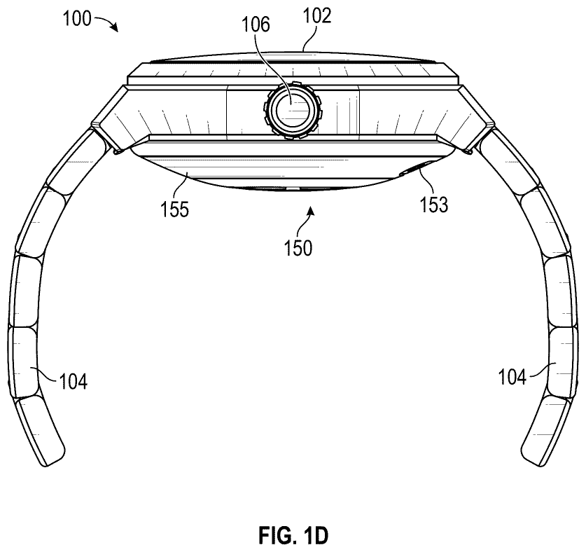

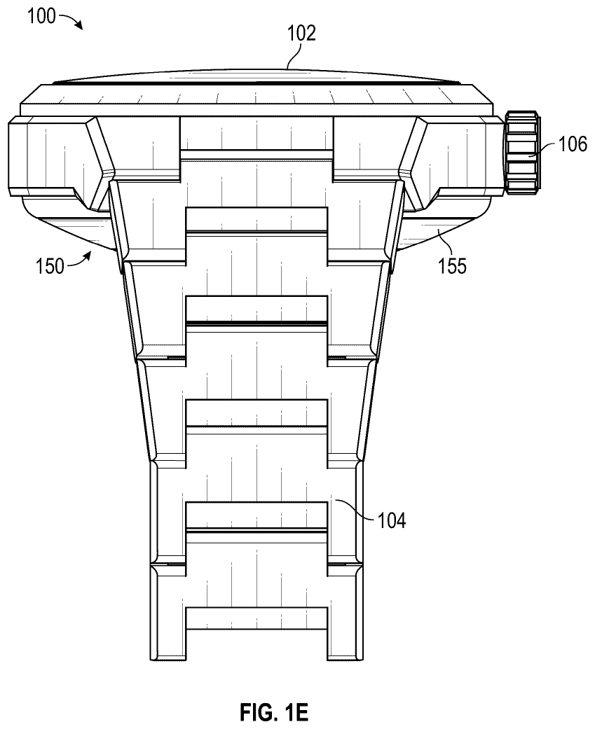

FIGS. 1A-1E illustrate various views of a wearable device 100 with a sensor assembly 150 coupled to the wearable device 100. Sensor assembly 150 can measure and/or monitor a user's physiological information, for example, oxygen saturation, heart rate, pulse rate, pulse rate variability, respiration rate, hydration index, and/or perfusion index. In some implementations, wearable device 100 is a watch, such as a mechanical watch, which can be mechanically powered. Wearable device 100 can include a face 102, an actuator 106, and/or straps 104. In some implementations, wearable device 100 can be worn on the wrist of a user. In some implementations, wearable device 100 can be worn on other parts of a user's body such as an ankle, leg, and/or arm. Portions of wearable device 100 can contact a user's tissue (e.g., skin) when wearable device 100 is worn by the user. Face 102 can be configured to indicate the time and/or the date to a user. In some implementations, face 102 can be a clock face, such as an analog clock face. Wearable device 100 can include a front portion that faces substantially away from a user's tissue (e.g., skin) when wearable device 100 is worn by the user. Wearable device 100 can include a back portion positioned substantially opposite the back portion of wearable device 100. Back portion of wearable device 100 can face substantially toward a user's tissue when wearable device 100 is worn by the user. Face 102 can be disposed on the front portion of wearable device 100 such that face 102 faces substantially away from a user's tissue and/or does not contact a user's tissue when wearable device 100 is worn by user.

Actuator 106 can be rotated to adjust the time and/or date indicated by face 102. Actuator 106 can be rotated to wind and/or power wearable device 100. In some implementations, actuator 106 can be a watch crown. Straps 104 can be configured to at least partially surround a user's wrist to secure wearable device 100 to a user's wrist. In some implementations, straps 104 can be configured to at least partially surround other parts of a user's body such as an ankle, leg, and/or arm such that wearable device 100 can be worn on other parts of a user's body. In some implementations, straps 104 can hold the portions of wearable device 100 (for example, the back portion of wearable device 100) and/or sensor assembly 150 against a user's tissue when wearable device 100 is worn by the user.

In some implementations, wearable device 100 does not include an electronic display. In some implementations, wearable device 100 does not include a battery. In some implementations, wearable device 100 does not include any electronic components. In some implementations, wearable device 100 is solely mechanically powered. In some implementations, wearable device 100 does not need to be electrically charged to keep time and/or display time.

Sensor assembly 150 can mechanically connect with wearable device 100. In some implementations, sensor assembly 150 can removably mechanically connect with wearable device 100 which can advantageously permit a user to wear wearable device 100 with or without sensor assembly 150 attached, for example, if a user wishes to wear wearable device 100 while charging sensor assembly 150. In some implementations, sensor assembly 150 can operate independently of wearable device 100 whether or not sensor assembly 150 is mechanically connected with wearable device 100. In some implementations, there is no electrical connection between wearable device 100 and sensor assembly 150 which can advantageously allow wearable device 100 to remain without electrical components to improve the appearance and/or longevity of wearable device 100. Providing no electrical connection between wearable device 100 and sensor assembly 150 can simplify the connection between wearable device 100 and sensor assembly 150 such that sensor assembly 150 can be more easily connected and/or disconnected from wearable device 100. Sensor assembly 150 can be rotated in a first direction to mechanically connect sensor assembly 150 to wearable device 100. In some implementations, sensor assembly 150 can be rotated in a second direction to mechanically disconnect sensor assembly 150 from wearable device 100. In some implementations, the second direction is substantially opposite the first direction. Allowing sensor assembly 150 to be selectively disconnected from wearable device 100 can advantageously allow a user to more easily access sensor assembly 150 for repairs and/or allow a user to replace sensor assembly 150 without having to replace wearable device 100. In some implementations, sensor assembly 150 can magnetically connect to wearable device 100.

Sensor assembly 150 can connect to wearable device 100 such that at least a portion of sensor assembly 150 faces toward a user's tissue and/or contacts a user's tissue when wearable device 100 is worn by the user. In some implementations, sensor assembly 150 can connect to the back portion of wearable device 100. Wearable device 100 can hold sensor assembly 150 against a user's tissue when sensor assembly 150 is connected to wearable device 100 and wearable device 100 is worn by the user. Holding sensor assembly 150 against a user's tissue can advantageously allow sensor assembly 150 to more accurately and/or consistently measure and/or monitor a user's physiological information.

Sensor assembly 150 can be circular or substantially circular. Sensor assembly 150 can have a curved (e.g., convex) surface. The curved surface of sensor assembly 150 can be positioned on a bottom portion of sensor assembly 150. Sensor assembly 150 can connect to wearable device 100 such that the curved surface of sensor assembly 150 faces substantially away from the bottom portion of wearable device 100. Sensor assembly 150 can connect to wearable device 100 such that the curved surface faces substantially toward a user's tissue and/or contacts a user's tissue when wearable device 100 is worn by the user. The curved surface can advantageously improve user comfort where sensor assembly 150 contacts the user's tissue.

Sensor assembly 150 can include one or more windows 151, one or more charging ports 153, and/or a shroud 155. The one or more windows 151 can be positioned on sensor assembly 150 such that the one or more windows 151 face substantially toward a user's tissue and/or contact a user's tissue when sensor assembly 150 is connected to wearable device 100 and wearable device 100 is worn by the user. The one or more windows 151 can be positioned on the bottom surface of sensor assembly 150. The one or more windows 151 can be positioned on the curved surface of sensor assembly 150. In some implementations, the one or more windows 151 can form a portion of the curved surface of sensor assembly 150. In some implementations, the one or more windows 151 can be embedded within the curved surface of sensor assembly 150 that is configured to contact the user's skin when the wearable device 100 is worn with the sensor assembly 150. The one or more windows 151 can be transparent and/or semi-transparent such that optical radiation can be transmitted through the one or more windows 151. In some implementations, one or more sensors can be positioned behind the one or more windows 151 such that the one or more sensors can emit and receive information through the one or more windows 151. In some implementations, sensor assembly 150 can include a plurality of windows 151.

The one or more charging ports 153 can be configured to receive energy to charge sensor assembly 150. The one or more charging ports 153 can be positioned on sensor assembly 150, which can advantageously permit sensor assembly 150 to be charged whether sensor assembly 150 is connected or disconnected from wearable device 100, for example, when a user wants to wear wearable device 100 while charging sensor assembly 150. Positioning the one or more charging ports 153 on sensor assembly 150, rather than on wearable device 100, can allow wearable device 100 to not include any electronic components. The one or more charging ports 153 can be positioned on sensor assembly 150 such that the one or more charging ports 153 face substantially away from the bottom portion of wearable device 100 when sensor assembly 150 is connected to wearable device 100 which can advantageously permit user access to the one or more charging ports 153 when sensor assembly 150 is connected to wearable device 100, for example, to charge sensor assembly 150. The one or more charging ports can be positioned on the bottom portion of sensor assembly 150. In some implementations, the one or more charging ports 153 can form a portion of the curved surface of sensor assembly 150. In some implementations, the one or more charging ports 153 can be embedded within the curved surface of sensor assembly 150 that is configured to contact the user's skin when the wearable device 100 is worn with the sensor assembly 150.

Shroud 155 can be positioned on sensor assembly 150 such that shroud 155 faces substantially toward and/or contacts a user's tissue when sensor assembly 150 is connected to wearable device 100 and wearable device 100 is worn by the user. In some implementations, shroud 155 can be circular and/or annular in shape. In some implementations, shroud 155 can form a portion of the curved surface of sensor assembly 150. In some implementations, shroud 155 can be embedded within the curved surface of sensor assembly 150 that is configured to contact the user's skin when the wearable device 100 is worn with the sensor assembly 150. In some implementations, shroud 155 can surround the one or more windows 151 when connected to sensor assembly 150. In some implementations, shroud 155 can have a different color and/or texture than sensor assembly 150. In some implementations, shroud 155 can have a texture and/or color that is the same or substantially similar to the texture and/or color of portions of wearable device 100, for example, straps 104 and/or actuator 106. In some implementations, shroud 155 can be made of the same material as portions of wearable device 100, for example, straps 105 and/or actuator 106. Shroud 155 can be removably connected to sensor assembly 150 which can advantageously allow a user to easily replace and/or customize shroud 155. For example, a user can replace shroud 155 with a shroud 155 that matches the material, texture, and/or color of wearable device 100.

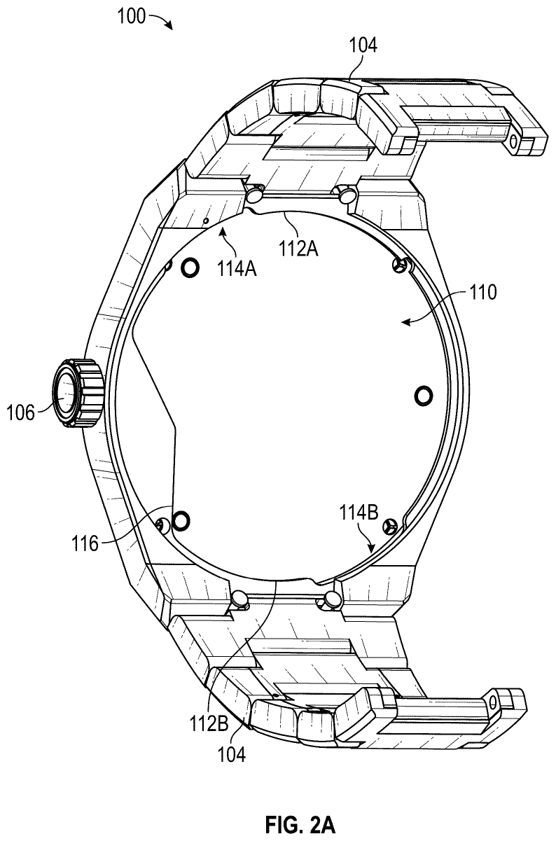

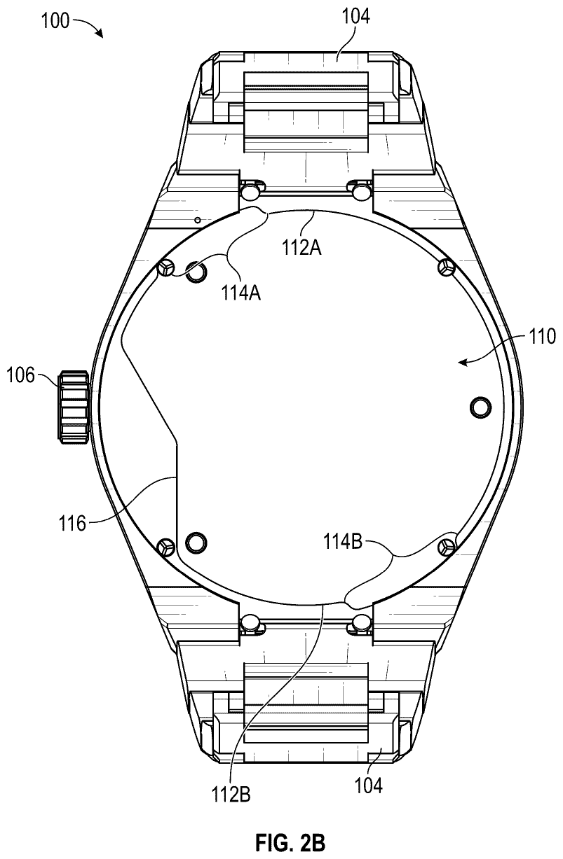

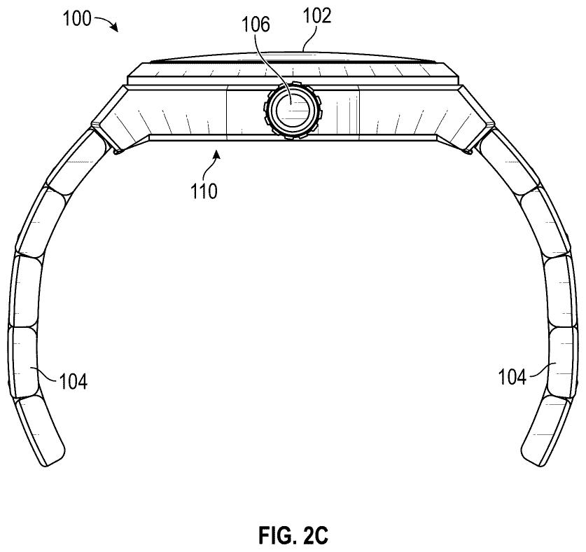

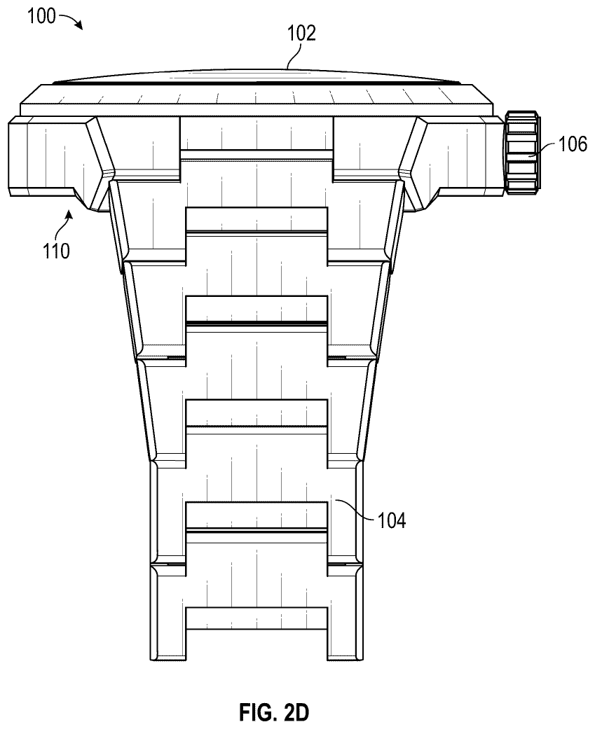

FIGS. 2A-2D illustrate various views of wearable device 100 configured to receive sensor assembly 150. Wearable device 100 can include a receptacle 110, one or more rails 112A, 112B, one or more openings 114A, 144B, and/or a barrier 116. Receptacle 110 can removably mechanically connect to sensor assembly 150 to connect sensor assembly 150 to wearable device 100. Receptacle 110 can receive a portion of sensor assembly 150 such that a portion of sensor assembly 150 is seated in receptacle 110 when sensor assembly 150 is mechanically connected to receptacle 110. Seating a portion of sensor assembly 150 within receptacle 110 can reduce the amount by which sensor assembly 150 protrudes from receptacle 110 when connected, which can advantageously reduce the footprint of sensor assembly 150 when connected to wearable device 100. In some implementations, sensor assembly 150 can be substantially flush with a surface of wearable device 100 when sensor assembly 150 is connected to receptacle 110 which can advantageously improve user comfort where wear wearable device 100 and/or sensor assembly 150 contact a user's tissue.

Sensor assembly 150 can be rotated relative to receptacle 110 to mechanically connect and/or disconnect sensor assembly 150 to receptacle 110. Sensor assembly 150 can be rotated in the first direction (for example, clockwise) to mechanically connect sensor assembly 150 to receptacle 110. Sensor assembly 150 can be rotated in the first direction to a desired orientation relative to receptacle 110 and/or wearable device 100. Sensor assembly 150 can be rotated in the second direction (for example, counter-clockwise) to mechanically disconnect sensor assembly 150 from receptacle 110. In some implementations, the second direction can be opposite the first direction. Receptable 110 can hold sensor assembly 150 against a user's tissue when sensor assembly 150 is mechanically connected to receptacle 110 and wearable device 100 is worn by the user. Receptacle 110 can be positioned on wearable device 100 such that receptacle 110 faces substantially toward a user's tissue when wearable device 100 is worn by the user. Receptacle 110 can be positioned on the back portion of wearable device 100. Receptacle 110 can be a recess sized and shaped such that at least a portion of sensor assembly 150 can be seated in receptacle 110. In some implementations, receptable 110 can be at least partially circular in shape. In some implementations, receptacle 110 can have a maximum diameter that is less than the maximum diameter of sensor assembly 150 such that a portion of sensor assembly 150 covers receptacle 110 when sensor assembly 150 is connected to receptacle 110. In some implementations, receptacle 110 can be magnetic and/or include a magnetic material such that receptacle 110 can magnetically connect to sensor assembly 150.

In some implementations, receptable 110 can removably mechanically connect with a cover when sensor assembly 150 is mechanically disconnected from receptable 110. Receptacle 110 can hold the cover against a user's tissue when the cover is mechanically connected to receptacle 110 and wearable device 100 is worn by the user. The cover can provide a smooth interface between wearable device 100 and a user's tissue if a user wants to wear wearable device 100 when sensor assembly 150 is disconnected from wearable device 100, for example, when a user wants to wear wearable device 100 while charging sensor assembly 150.

The one or more rails 112A, 112B can mechanically mate with sensor assembly 150 to hold sensor assembly 150 to receptable 110 and/or wearable device 100. The one or more rails 112A, 112B can be positioned on the back portion of wearable device 100. The one or more rails 112A, 112B can be positioned within receptacle 110. The one or more rails 112A, 112B can have a length that extends at least partially along an outer edge of receptacle 110. The one or more rails 112A, 112B can be curved to conform to the shape of the outer edge of receptable 110. In some implementations, the one or more rails 112A, 112B can curve along a circular path, for example, if receptacle 110 is at least partially circular in shape. Rail 112A and rail 112B can have different lengths. Rail 112A can be longer than rail 112B. In some implementations, rail 112A and rail 112B can have the same or substantially similar lengths.

The one or more rails 112A, 112B can have a width that protrudes inward from the outer edge of receptacle 110 toward a center of receptacle 110. The one or more rails 112B, 112B can have the same or substantially similar widths. In some implementations, the one or more rails 112A, 112B can have different widths. Rail 112A can be positioned above a bottom surface of receptacle 110 such that there is a recess between rail 112A and the bottom surface of receptacle 110. In some implementations, a portion of sensor assembly 150 can be inserted into the recess between rail 112A and receptacle 110 to mechanically connect sensor assembly 150 to receptacle 110 and wearable device 100. In some implementations, a portion of sensor assembly 150 can slide into the recess between rail 112A and receptacle 110 when sensor assembly 150 is rotated in the first direction. In some implementations, rail 112B can be positioned above the bottom surface of receptacle 110 such that there is a recess between rail 112B and the bottom surface of receptacle 110. In some implementations, a portion of sensor assembly 150 can be inserted into the recess between rail 112B and receptacle 110 to mechanically connect sensor assembly 150 to receptacle 110 and wearable device 100. In some implementations, a portion of sensor assembly 150 can slide into the recess between rail 112A and receptacle 110 when sensor assembly 150 is rotated in the first direction. In some implementations, wearable device 100 can include two rails 112A, 112B. In some implementations, wearable device 100 can include only one of rail 112A, 112B.

The one or more openings 114A, 114B can receive portions of sensor assembly 150 when sensor assembly 150 is connected and/or disconnected from wearable device 100. In some implementations, one or more portions of sensor assembly 150 can be inserted through the one or more openings 114A, 114B when sensor assembly 150 is connected to receptable 110 and/or one or more portions of sensor assembly 150 can be extracted through the one or more openings 114A, 114B when sensor assembly is disconnected from receptacle 110. The one or more openings 114A, 114B can be positioned on the back portion of the wearable device 100. The one or more openings 114A, 114B can be positioned within receptacle 110. In some implementations, opening 114A and/or opening 114B can separate rails 112A, 112B. In some implementations, opening 114A and/or opening 114B can be a gap between rails 112A, 112B. In some implementations, opening 114A and opening 114B can be positioned substantially opposite each other within receptacle 110. Openings 114A, 114B can be the same or substantially similar in size and/or shape. In some implementations, opening 114A and opening 114B can have different sizes and/or shapes. In some implementations, wearable device can include two openings 114A, 114B. In some implementations, wearable device 100 can include only one of opening 114A and opening 114B.

Barrier 116 can inhibit and/or prevent sensor assembly 150 from rotating beyond a certain orientation relative to receptacle 110 and wearable device 100. In some implementations, barrier 116 can contact a portion of sensor assembly 150 to inhibit and/or prevent sensor assembly from over rotating in the first direction, for example, when sensor assembly 150 is mechanically connected to wearable device 100. Preventing over rotation of sensor assembly 150 can advantageously allow sensor assembly 150 to have a consistent rotational orientation relative to wearable device 100 when fully connected to wearable device 100. In some implementations, barrier 116 can contact a portion of sensor assembly 150 to inhibit and/or prevent sensor assembly from over rotating in the second direction, for example, when sensor assembly 150 is mechanically disconnected from wearable device 100. In some implementations, barrier 116 can include a planar surface. Barrier 116 can be positioned on the back portion of wearable device 100. Barrier 116 can be positioned within receptacle 110. Barrier 116 can extend from an outer edge or receptacle 110 into receptacle 110. In some implementations, barrier 116 can be positioned adjacent rail 112B. In some implementations, barrier 116 can extend from rail 112B.

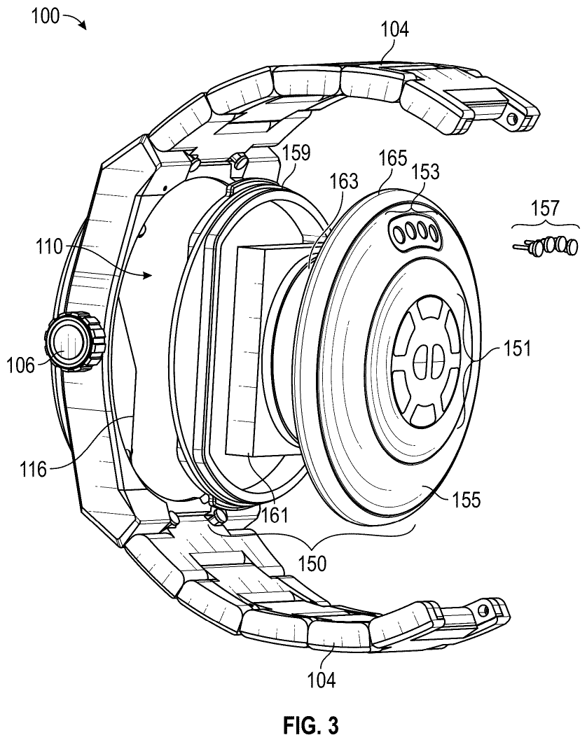

FIG. 3 illustrates a perspective view of the wearable device 100 with an exploded view of sensor assembly 150. Sensor assembly 150 can include a back housing 159, a battery 161, one or more charging pins 157, a circuit board 163, and/or a front housing 165. The term “housing” can be used herein to refer to back housing 159 and/or front housing 165 separately or in combination.

Back housing 159 can removably mechanically connect to wearable device 100. In some implementations, back housing 159 can removably mechanically connect to receptacle 110. In some implementations, sensor assembly 150 can removably connect to receptacle 110 and/or wearable device 100 through back housing 159. Back housing 159 can be shaped and sized such that a portion of back housing 159 can be seated in receptacle 110 when back housing 159 is mechanically connected to receptacle 110. In some implementations, one or more portions of back housing 159 can be annular, ovoid, and/or circular in shape. In some implementations, back housing 159 can include a recessed portion shaped and sized to at least partially house battery 161 and/or circuit board 163.

Back housing 159 can be rotated relative to receptacle 110 to mechanically connect and/or disconnect back housing 159 and/or sensor assembly 150 to receptacle 110 and/or wearable device 100. Sensor assembly 150 can be configured such that some or all the components of sensor assembly 150 rotate with back housing 159. Back housing 159 can be rotated in the first direction to mechanically connect back housing 159 and/or sensor assembly 150 to receptacle 110 and/or wearable device 100. In some implementations, back housing 159 can be rotated in the first direction to a desired orientation relative to receptacle 110. In some implementations, a portion of back housing 159 can contact barrier 116 to inhibit and/or prevent back housing 159 from over rotating in the first direction. Back housing 159 can be rotated in the second direction to mechanically disconnect back housing 159 and/or sensor assembly 150 from receptacle 110 and/or wearable device 100. In some implementations, a portion of back housing 159 can contact a portion of receptacle 110, such as barrier 116, to prevent back housing 159 from over rotating in the second direction.

In some implementations, a portion of back housing 159 can be inserted into the recess between rail 112A and receptacle 110 to mechanically connect back housing 159 and/or sensor assembly 150 to receptacle 110 and/or wearable device 100. In some implementations, a portion of back housing 159 can slide into the recess between rail 112A and receptacle 110 when back housing 159 and/or sensor assembly 150 is rotated in the first direction. In some implementations, a portion of back housing 159 can be inserted into the recess between rail 112B and receptacle 110 to mechanically connect back housing 159 and/or sensor assembly 150 to receptacle 110 and/or wearable device 100. In some implementations, a portion of back housing 159 can slide into the recess between rail 112B and receptacle 110 when back housing 159 and/or sensor assembly 150 is rotated in the first direction. In some implementations, back housing 159 can be magnetic and/or include a magnetic material such that back housing 159 can magnetically connect to wearable device 100 and/or receptacle 110.

Battery 161 can act as a power source for components of sensor assembly 150. Battery 161 can be electrically isolated from wearable device 100 such that battery 161 does not provide power to wearable device 100. Battery 161 can include a dual-battery configuration with a main battery and a backup battery. Battery 161 can include a lithium-based battery such as a lithium ion battery and/or a lithium polymer battery. Battery 161 can connect to an external power source to receive power from the external power source. In some implementations, battery 161 can be electrically connected to the one or more charging pins 157 such that battery 161 can connect to an external power source through charging pins 157. Battery 161 can be electrically connected to circuit board 163 such that battery 161 provides power to circuit board 163. Battery 161 can be rotatably coupled to back housing 159. In some implementations, battery 161 can be positioned between back housing 159 and front housing 165. Battery 161 can be housed by back housing 159 and/or front housing 165.

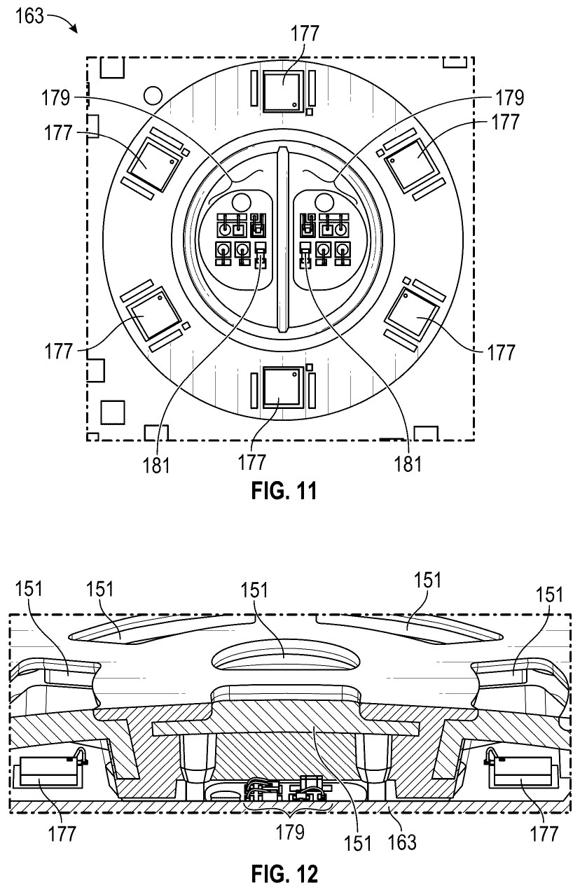

Circuit board 163 can house various electronic components of the sensor assembly 150 as further described below with respect to FIGS. 11-12. Circuit board 163 can receive power from battery 161. Circuit board 163 can be positioned on battery 161 such that it faces substantially toward front housing 165 and/or the one or more windows 151. Circuit board 163 can be rotatably coupled to back housing 159. In some implementations, circuit board 163 can be positioned between back housing 159 and front housing 165. Circuit board 163 can be housed by back housing 159 and/or front housing 165.

Front housing 165 can at least partially enclose circuit board 163 and/or battery 161. Front housing 165 can mechanically connect to back housing 159. In some implementations, front housing 165 can removably mechanically connect to back housing 159. In some implementations, front housing 165 and back housing 159 can be integrally formed such that they form a singular piece. In some implementations, battery 161 and/or circuit board 163 can be positioned between back housing 159 and front housing 165 such that battery 161 and circuit board 163 are surrounded by back housing 159 and front housing 165.

In some implementations, front housing 165 can be rotatably coupled to back housing 159. Front housing 165 can be circular or substantially circular in shape. Front housing 165 can include a curved surface that contacts tissue of a user when sensor assembly 150 is connected to wearable device 100 and wearable device 100 is worn by the user. In some implementations, the one or more windows 151 and/or the one or more charging ports 153 can be embedded in front housing 165. In some implementations, the one or more windows and/or the one or more charging ports 153 form a part of the curved surface of front housing 165. In some implementations, shroud 155 can be removably connected to front housing 165. In some implementations, shroud 155 forms a part of the curved surface of front housing 165 when shroud 155 is connected to front housing 165.

The one or more charging pins 157 can be positioned on sensor assembly 150 and/or front housing 165. The one or more charging pins 157 can be housed by one or more charging ports 153. In some implementations, the one or more charging pins 157 can be positioned on or beneath a curved surface of the front housing 165, such as a curved surface configured to contact the skin of the user. In some implementations, the one or more charging pins 157 can be recessed from an outer surface of the one or more charging ports 153 and/or the curved surface of sensor assembly 150 such that the one or more charging pins 157 do not contact a user's tissue when sensor assembly 150 is connected to wearable device 100 and wearable device 100 is worn by the user. In some implementations, the one or more charging pins 157 can form part of the curved surface of front housing 165. In some implementations, the one or more charging pins 157 are not electrically connected to wearable device 100 or any components thereof, at least because wearable device 100 may not of itself include any electrical components (other than removable sensor assembly 150), such as a battery or electronic display. In some implementations, the one or more charging pins 157 are removable from wearable device 100, for example, when sensor assembly 150 is removed from wearable device 100. This can advantageously allow the sensor assembly 150 to be charged even when disconnected from wearable device 100.

In some implementations, sensor assembly 150 does not include electrodes which can reduce the amount of area needed for front housing 165. In some implementations, sensor assembly 150 does not include inductive charging coils for wireless charging which can advantageously reduce the size of sensor assembly 150.

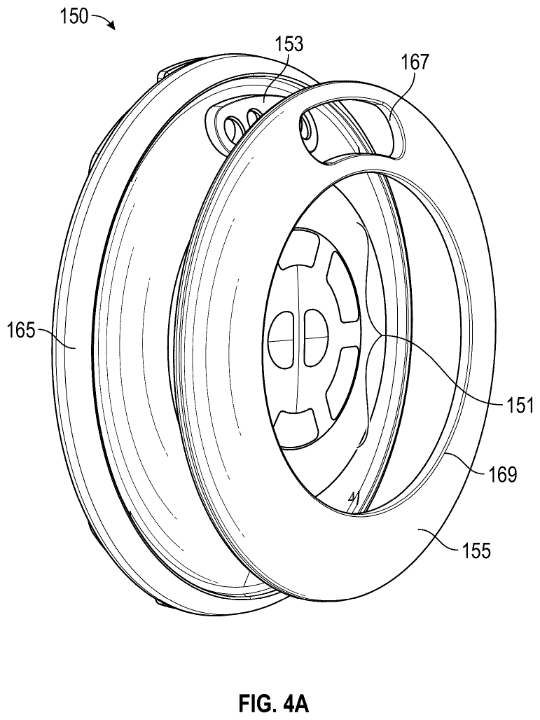

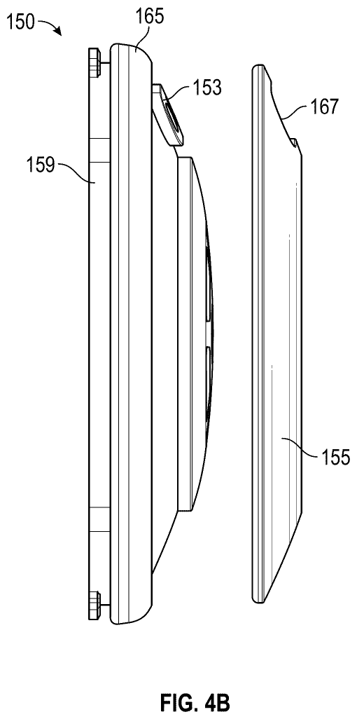

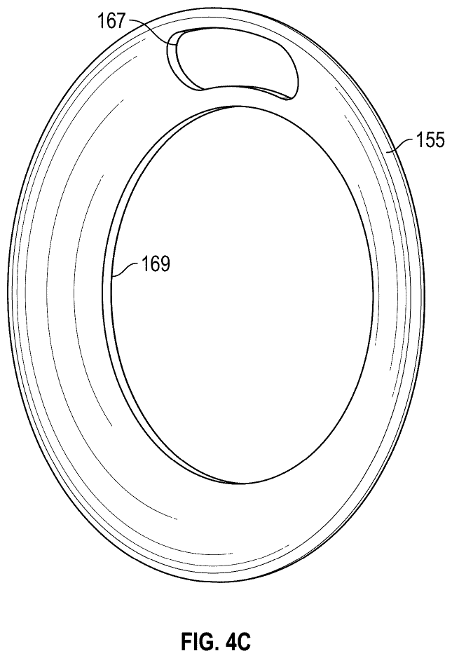

FIGS. 4A-4B illustrate exploded views of a portion of sensor assembly 150. FIG. 4C illustrates a perspective view of shroud 155 of sensor assembly 150. Front housing 165 can include a recessed portion shaped and sized to receive shroud 155. The recessed portion of front housing 165 can be annular in shape. Shroud 155 can removably connect to the recessed portion of front housing 165. In some implementations, shroud 155 can form an interference fit with the recessed portion of front housing 165. In some implementations, shroud 155 can be seated in the recessed portion of front housing such that shroud 155 is substantially flush with a curved surface of sensor assembly 150, for example, a curved surface of front housing 165. In some implementations, shroud 155 can be magnetic and/or include a magnetic material such that shroud 155 can magnetically connect to sensor assembly 150, for example, front housing 165. In some implementations, the recessed portion of front housing 165 can be magnetic and/or include a magnetic material such that the recessed portion of front housing 165 can magnetically connect to shroud 155.

Shroud 155 and front housing 165 can form a curved surface when shroud 155 is connected to front housing 165. In some implementations, shroud 155 and front housing 165 can form a substantially continuous surface when shroud 155 is connected to front housing 165. In some implementations, shroud 155 is substantially flush with front housing 165 when shroud 155 is connected to front housing 165 which can reduce the risk of pinching a user's tissue when sensor assembly 150 contacts the user's tissue. Shroud 155 can connect to front housing 165 such that shroud 155 can contact a user's tissue when sensor assembly 150 is connected to wearable device 100 and wearable device 100 is worn by the user.

Shroud 155 can include a sensor opening 169 and/or a port opening 167. Sensor opening 169 can be circular or substantially circular in shape. Sensor opening 169 can extend through shroud 155. Sensor opening 169 can be shaped and sized to surround the one or more windows 151 such that the one or more windows 151 can be inserted into sensor opening 169 when shroud 155 is connected to front housing 165. Providing an opening in shroud 155 to receive the one or more windows 151 can advantageously provide the one or more windows 151 with an unobstructed view of a user's tissue when sensor assembly 150 is connected to wearable device 100 and wearable device 100 is worn by a user. In some implementations, shroud 155 is substantially flush with windows 151 when shroud 155 is connected to front housing 165 which can advantageously improve user comfort when sensor assembly 150 contacts the user's tissue and/or reduce the risk of pinching the user's tissue. In some implementations, portions of front housing 165, shroud 155, and/or the one or more windows 151 can form a substantially continuous curved surface which can advantageously increase user comfort when sensor assembly 150 contacts a user's tissue.

Port opening 167 can be annular in shape. Port opening 167 can extend through shroud 155. Port opening 167 can be shaped and sized to surround the one or more charging ports 153 such that the one or more charging ports 153 can be inserted into port opening 167 when shroud 155 is connected to front housing 165. Providing an opening in shroud 155 to receive the one or more charging ports 153 can advantageously provide access to charging ports 153 such that sensor assembly 150 can be charged when shroud 155 is connected to front housing 165. In some implementations, shroud 155 is substantially flush with portions of the one or more charging ports 153 when shroud 155 is connected to front housing 165 which can advantageously improve user comfort when sensor assembly 150 contacts the user's tissue and/or reduce the risk of pinching the user's tissue.

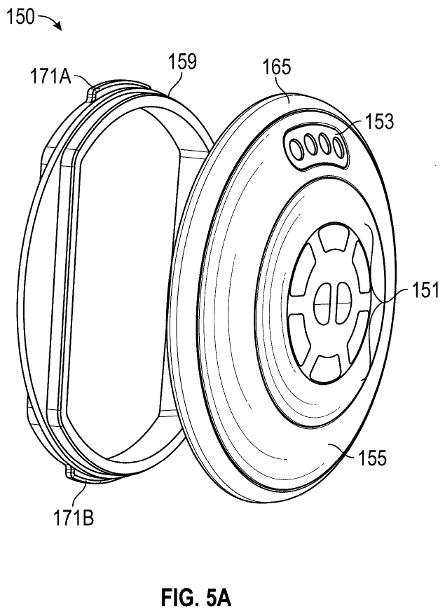

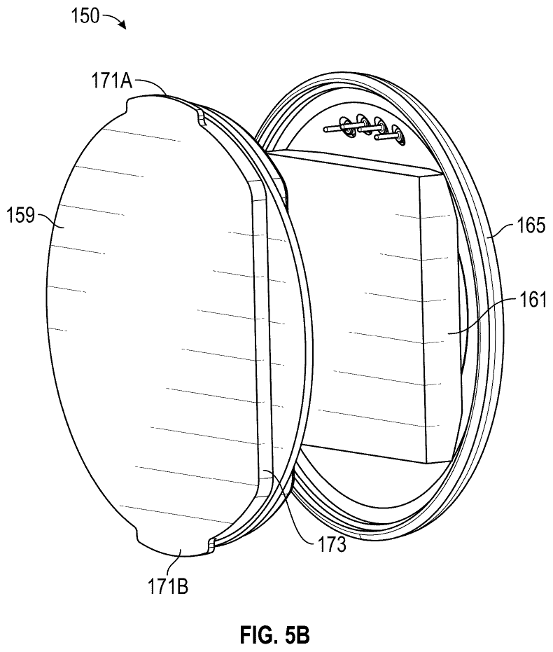

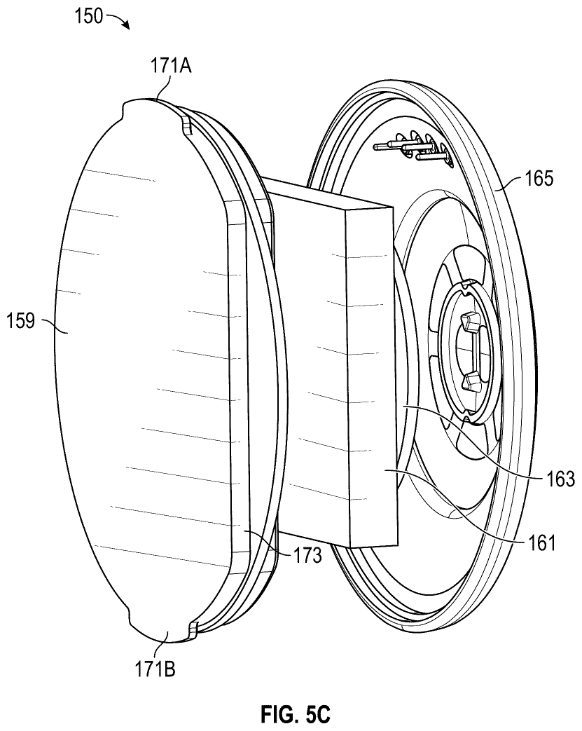

FIGS. 5A-5C illustrate exploded views of sensor assembly 150. Back housing 159 can include one or more ribs 171A, 171B and/or a surface (e.g., contact surface, wall, protrusion) 173. The one or more ribs 171A, 171B can slide within wearable device 100 to secure back housing 159 and/or sensor assembly 150 to wearable device 100. The one or more ribs 171A, 171B can extend from back housing 159, for example, an outer surface of back housing 159. The one or more ribs 171A, 171B can be positioned on a rear portion of back housing 159. In some implementations, ribs 171A, 171B can be positioned on opposite or substantially opposite ends of back housing 159. In some implementations, rib 171A can be positioned on a top end of back housing 159 and/or rib 171B can be positioned on a bottom end of back housing 159. In some implementations, the one or more ribs 171A, 171B can be curved. Ribs 171A, 171B can be the same or substantially similar in size and/or shape. In some implementations, ribs 171A, 171B can have different sizes and/or shapes. In some implementations, back housing 159 can include only one of rib 171A and rib 171B.

Surface 173 can inhibit and/or prevent back housing 159 and/or sensor assembly 150 from rotating beyond a certain orientation relative to receptacle 110 and wearable device 100. Surface 173 can contact a portion of wearable device 100 to inhibit and/or prevent sensor assembly 150 from over rotating in the first direction, for example, when sensor assembly 150 is connected to wearable device 100. In some implementations, surface 173 can contact a portion of wearable device 100 to inhibit and/or prevent sensor assembly 150 from over rotating in the second direction, for example, when sensor assembly is disconnected from wearable device 100. Surface 173 can be positioned on a lateral side of back housing 159. Surface 173 can be substantially planar. Surface 173 can be positioned adjacent a rear side of back housing 159. In some implementations surface 173 can be substantially perpendicular to rib 171A and/or rib 171B.

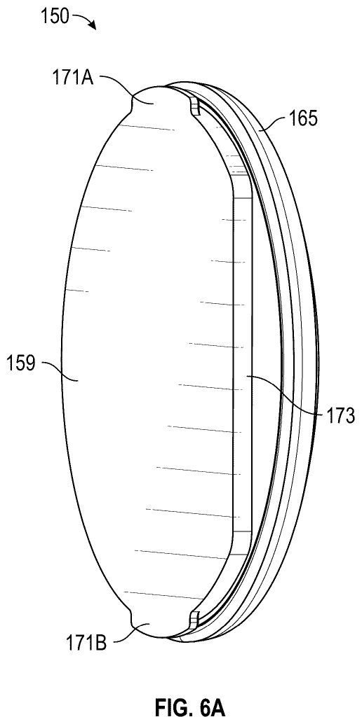

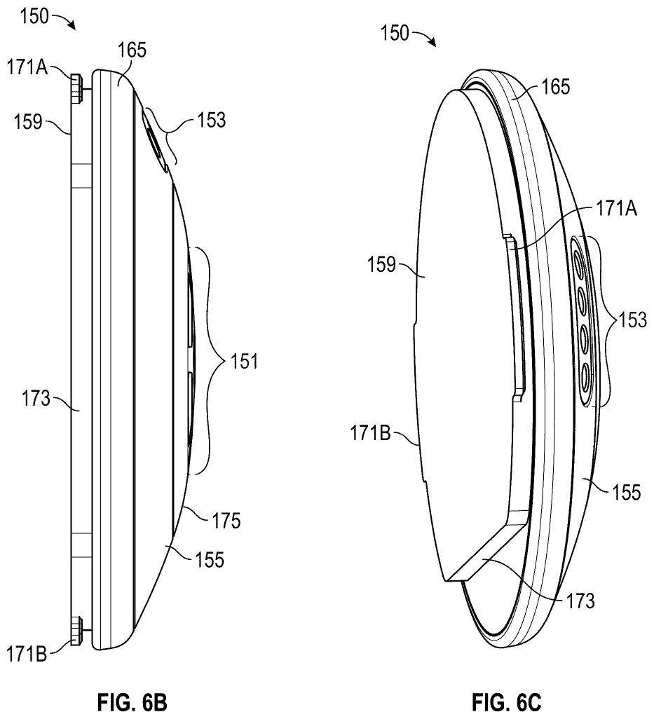

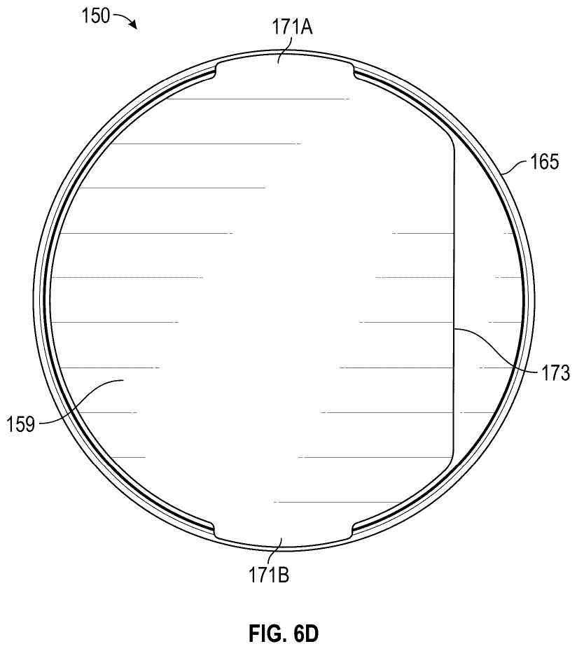

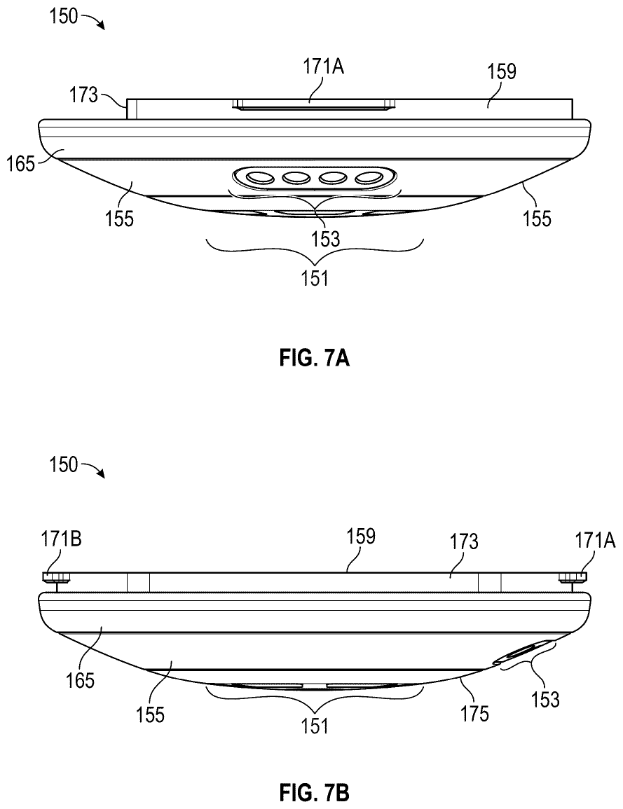

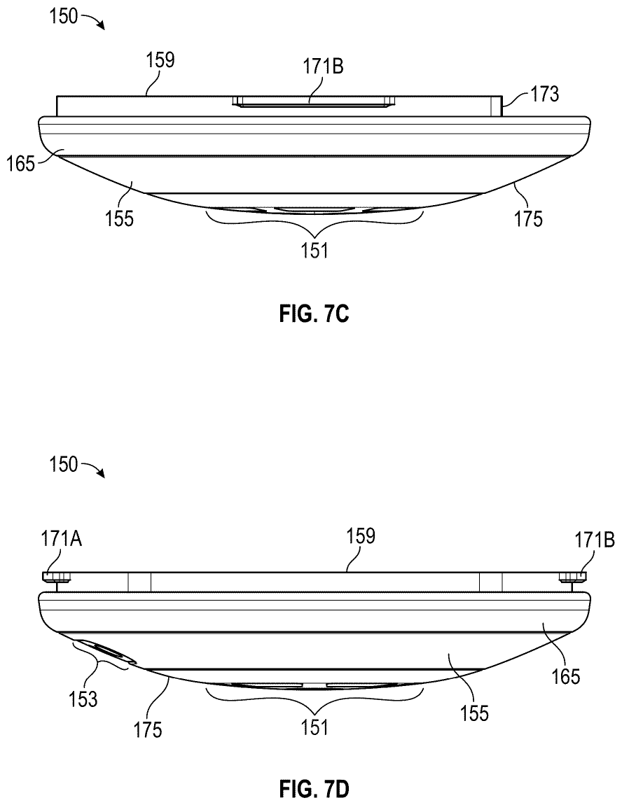

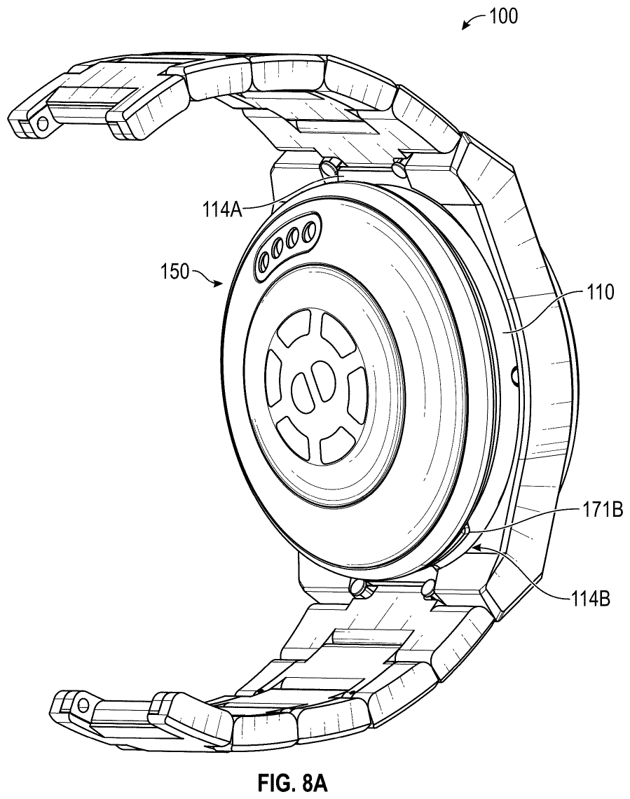

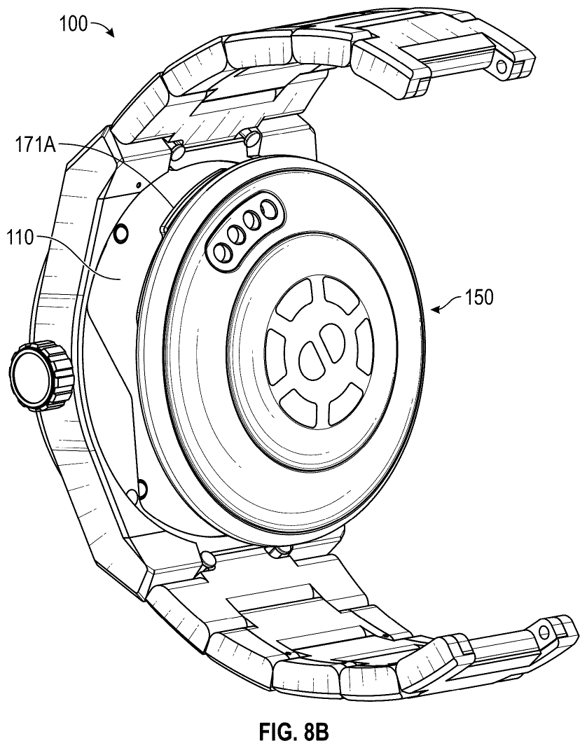

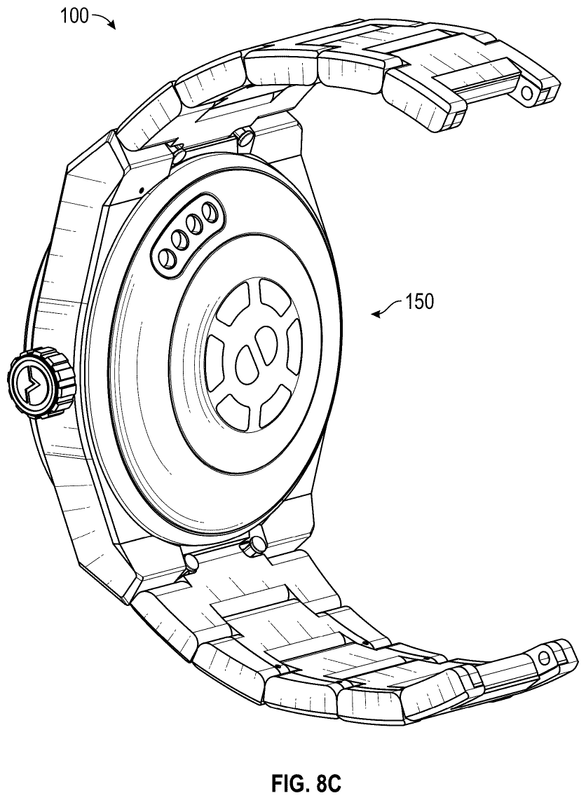

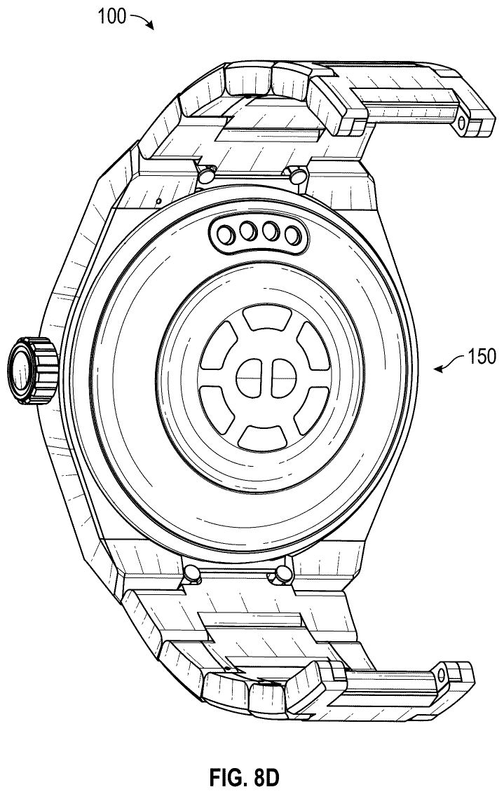

FIGS. 6A-7D illustrate various views of sensor assembly 150. Front housing 165 can mechanically connect to back housing 159. In some implementations, front housing 165 can removably connect to back housing 159. In some implementations, front housing 165 can be connected to back housing 159 with an interference fit. In some implementations, a portion of back housing 159 (for example, a front portion) can be inserted into front housing 165 when back housing 159 is connected to front housing 165 such that the portion of back housing 159 is positioned within front housing 165. This can advantageously reduce the size and/or footprint of sensor assembly 150. In some implementations, a rear portion of back housing 159 can protrude from front housing 165 when back housing 159 is connected to front housing 165. In some implementations, the rear portion of back housing 159 can be inserted at least partially into receptacle 110 when sensor assembly 150 is connected to wearable device 100 such that only a portion of wearable device 100 (for example, front housing 165) protrudes from receptacle 110 and/or wearable device 100 (see FIGS. 8A-8D). This can advantageously reduce the footprint of sensor assembly 150 and wearable device 100 when connected and/or reduce the visibility of sensor assembly 150 when connected to wearable device 100. In some implementations, front housing 165 can have a maximum diameter that is greater than a maximum diameter of back housing 159 (see FIG. 6D) such that front housing 165 can partially or substantially cover back housing 159 and/or receptacle 110 when sensor assembly 150 is connected to wearable device, for example, when back housing 159 is inserted into receptacle 110 (see FIG. 8D). This can advantageously reduce the risk of user tissue (e.g., skin, hair) catching in back housing 159 and/or receptacle 110 when sensor assembly 150 is connected to wearable device and wearable device 100 is worn by a user.

In some implementations, front housing 165 can be rotatably coupled to back housing 159 such that front housing 165 and back housing 159 rotate together. In some implementations, battery 161 and/or circuit board 163 can be housed between front housing 165 and back housing 159 such that battery 161 and/or circuit board 163 rotate with both back housing 159 and front housing 165. In some implementations, surface 173 can be positioned inward of a curved outer circumferential edge of front housing 165 when front housing 165 is connected to back housing 159. In some implementations, front housing 165 can connect to back housing 159 such that the one or more charging ports 153 are substantially or partially aligned with rib 171A. In some implementations, front housing 156 can connect to back housing 159 such that surface 173 is substantially perpendicular to the one or more charging ports 153.

FIGS. 8A-8D illustrate various views of sensor assembly 150 being connected to wearable device 100. An example method of mechanically connecting sensor assembly 150 to wearable device 100 is described below. First, sensor assembly 150 can be positioned adjacent receptacle 110 of wearable device 100 such that rib 171A is aligned with opening 114A and/or rib 171B is aligned with opening 114B as shown in FIGS. 8A and 8B. Next, a portion of sensor assembly 150 (for example, back housing 159) can be inserted at least partially into receptacle 110 such that rib 171A is inserted into receptacle 110 through opening 114A and/or rib 171B is inserted receptacle 110 through opening 114B as shown in FIG. 8C. Next, sensor assembly 150 can be rotated relative to wearable device 100 in the first direction (for example, clockwise) to mechanically connect sensor assembly 150 to wearable device 100 as shown in FIG. 8D. Sensor assembly 150 can be rotated in the first direction until a portion of sensor assembly 150 contacts a portion of wearable device 100 such that further rotation of sensor assembly 150 relative to wearable device 100 in the first direction is inhibited, which can be referred to as a fully connected position. FIG. 8D illustrates an example of the sensor assembly 150 in the fully connected position. In some implementations, the one or more charging ports 153 can be positioned adjacent a top side of wearable device 100 when sensor assembly 150 is in the fully connected position.

In some implementations, sensor assembly 150 can be selectively disconnected from wearable device 100, for example, if a user wishes to remove sensor assembly 150 for maintenance or use wearable device 100 without sensor assembly 150. An example method of mechanically disconnecting sensor assembly 150 from wearable device 100 is described below. First, sensor assembly 150 can be rotated in the second direction (for example, counter-clockwise) until rib 171A is aligned with opening 114A and/or rib 171B is aligned with opening 114B. In some implementations, a portion of sensor assembly 150 can contact a portion of wearable device 100 such that further rotation in the second direction is inhibited. Next, sensor assembly 150 can be disconnected from receptacle 110 and/or wearable device 100 by moving sensor assembly 150 away from receptacle 110 such that rib 171A is extracted from receptacle 110 through opening 114A and/or rib 171B is extracted from receptacle 110 through opening 114B.

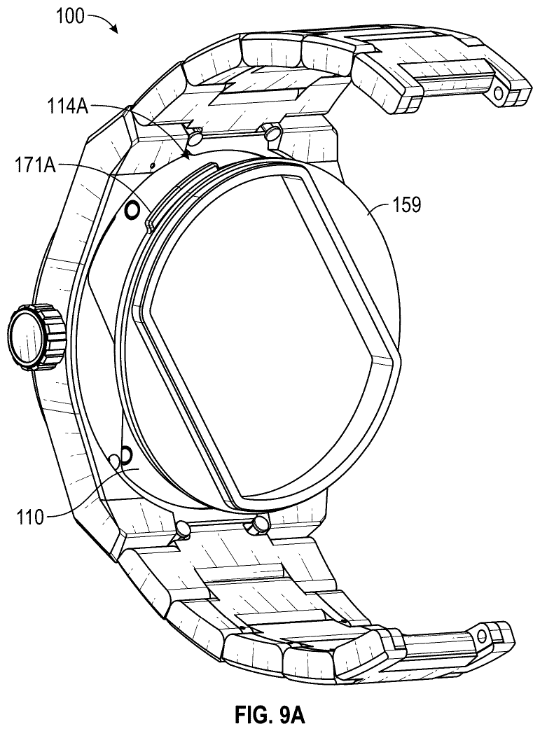

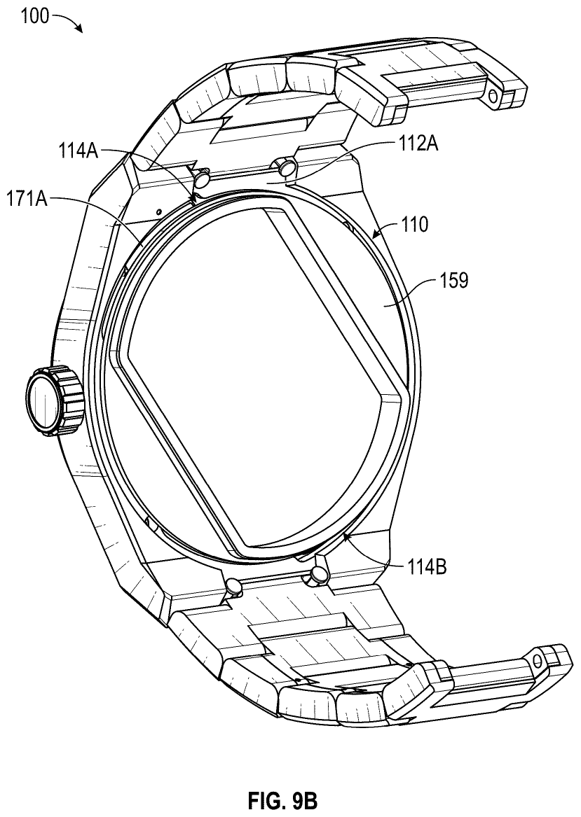

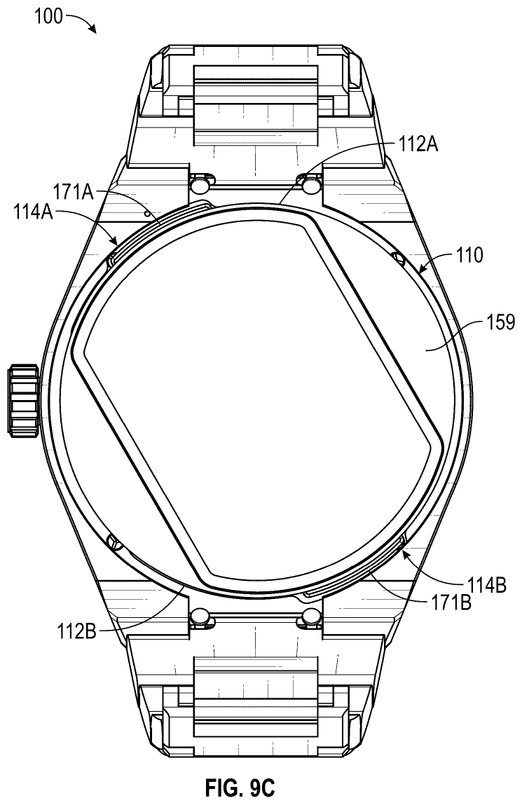

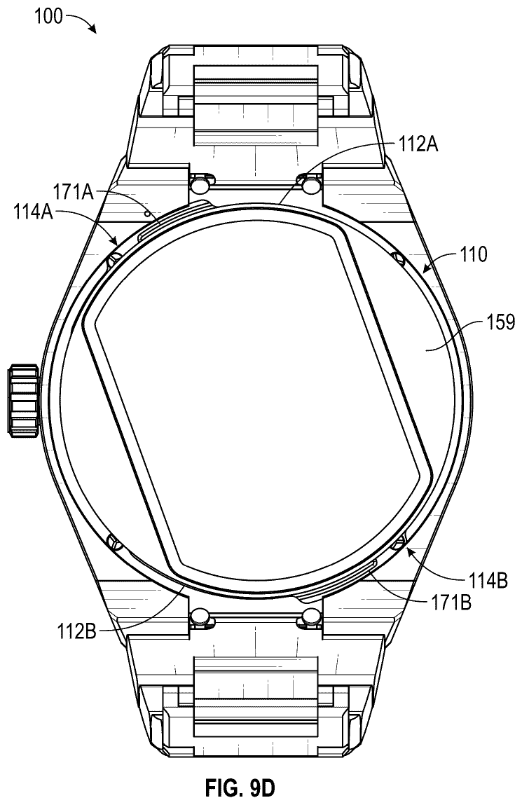

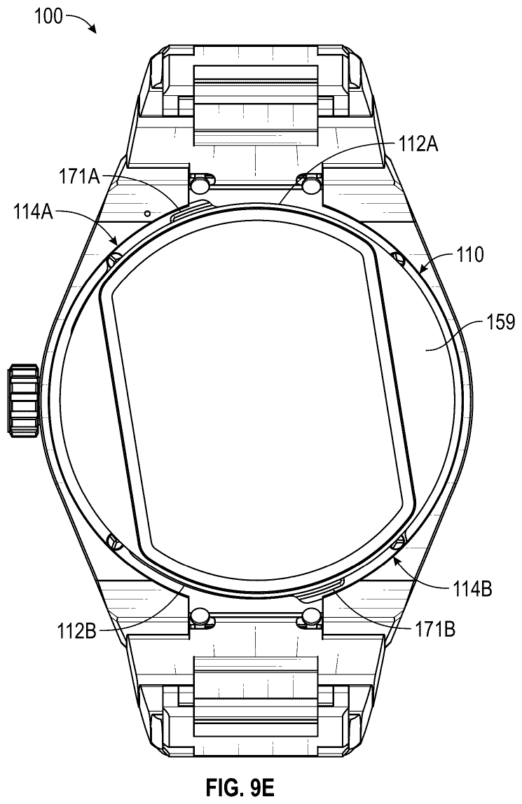

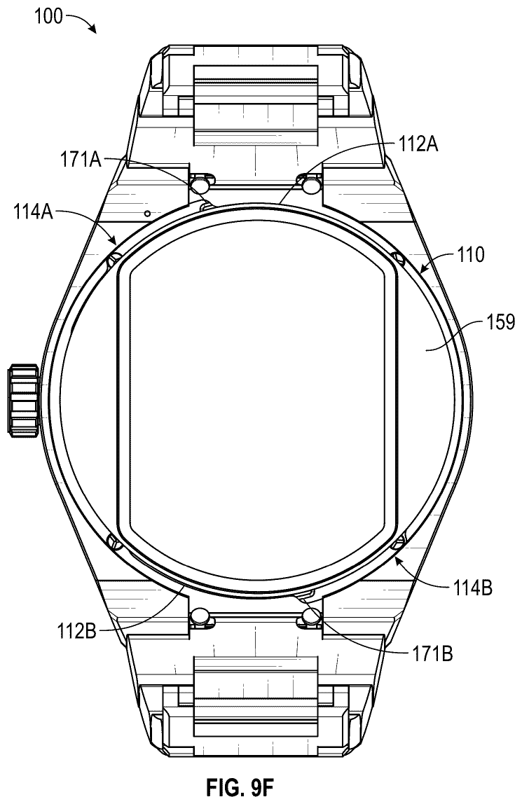

FIGS. 9A-9F illustrate various views of back housing 159 of sensor assembly 150 being connected to wearable device 100 for purposes of clarity. It will be understood that other components of sensor assembly 150 (for example, front housing) can be connected and/or rotatably coupled to back housing 159 such that sensor assembly 150 as a whole can be connected to wearable device 100 when back housing 159 is connected to wearable device 100 and disconnected from wearable device 100 when back housing is disconnected from wearable device 100. An example method of mechanically connecting back housing 159, and thus sensor assembly 150, to wearable device 100 is described below.

First, back housing 159 can be positioned adjacent receptacle 110 of wearable device 100 such that rib 171A is aligned with opening 114A and/or rib 171B is aligned with opening 114B as shown in FIG. 9A. Next, back housing 159 can be inserted at least partially into receptacle 110 such that rib 171A is inserted into receptacle 110 through opening 114A and/or rib 171B is inserted into receptacle 110 through opening 114B as shown in FIGS. 9B-9C. Next, back housing 159 can be rotated relative to wearable device 100 in the first direction (for example, clockwise) as shown by FIGS. 9D-9F such that back housing 159 is mechanically connected to wearable device 100, for example, through rib 171A mechanically mating with rail 112A and/or rib 171B mechanically mating with rail 112B. In some implementations, back housing 159 can be rotated in the first direction until a portion of back housing 159 (for example, surface 173) contacts a portion of wearable device 100 (for example, barrier 116) such that further rotation of back housing 159 relative to wearable device 100 in the first direction is inhibited. This position can be referred to as the fully connected position. FIG. 9F illustrates an example of back housing 159 in the fully connected position.

In some implementations, rib 171A and/or rib 171B can rotate with back housing 159. As back housing 159 is rotated in the first direction, rib 171A can rotate in the first direction such that rib 171A slides at least partially into the recess positioned between rail 112A and the bottom surface of receptacle 110 and/or rib 171B can rotate in the first direction such that rib 171B slides at least partially into the recess positioned between rail 112B and the bottom surface of receptacle 110. In some implementations, rail 112A and/or rail 112B can inhibit back housing 159 from being removed from receptacle 110 such that back housing 159 remains connected to receptacle 110 and/or wearable device 100.

In some implementations, rail 112A can inhibit movement of rib 171A away from receptacle 110 and/or wearable device 100 by contacting rib 171A and/or rail 112B can inhibit movement of rib 171B away from receptacle 110 and/or wearable device 100 by contacting rib 171B, thereby inhibiting movement of back housing 159 and sensor assembly 150 away from receptacle 110 and/or wearable device 100. In some implementations, rail 112A can substantially limit movement of rib 171A and/or rail 112B can substantially limit movement of rib 171B to rotation in the first and/or second direction, thereby substantially limiting movement of back housing 159 to rotation in the first and/or second direction. By inhibiting movement of back housing 159 away from receptacle 110 and/or wearable device 100, rail 112A and/or rail 112B can hold back housing 159 against the wearable device 100 such that sensor assembly 150 remains mechanically connected to wearable device 100 until a user wishes to disconnect sensor assembly 150 from wearable device 100.

In some implementations, back housing 159 can be selectively disconnected from wearable device 100. An example method of mechanically disconnecting back housing 159 from wearable device 100 is described below. First, back housing 159 can be rotated in the second direction until rib 171A is aligned with opening 114A and/or rib 171B is aligned with opening 114B. In some implementations, back housing 159 is inhibited from over rotating in the second direction to prevent rib 171A rotating past alignment with opening 114A and/or to prevent rib 171B rotating past alignment with opening 114B. In some implementations, a portion of back housing 159 (for example, surface 173) can contact a portion of wearable device 100 (for example, barrier 116) to prevent over rotation in the second direction. Next, back housing 159 can be disconnected from receptacle 110 by moving back housing 159 away from receptacle 110 such that rib 171A is extracted from receptacle 110 through opening 114A and/or rib 171B is extracted from receptacle 110 through opening 114B.

In some implementations, the recess between rail 112A and the bottom surface of receptacle 110 can be sized such that rib 171A contacts rail 112A and/or the bottom surface of receptacle 110 when rib 171A is positioned at least partially in the recess. This contact can create a first frictional force between rib 171A and one or both of rail 112A and the bottom surface of receptacle 110 when rib 171A is rotated. In some implementations, the recess between rail 112B and the bottom surface of receptacle 110 can be sized such that rib 171B contacts rail 112B and/or the bottom surface of receptacle 110 when rib 171B is positioned at least partially in the recess. This contact can create a second frictional force between rib 171B and one or both of rail 112B and the bottom surface of receptacle 110 when rib 171B is rotated. The first and/or second frictional force can increase the force required to rotate back housing 159 in the second direction (for example, when disconnecting back housing 159 from receptacle 110) which can advantageously decrease the likelihood of sensor assembly 150 inadvertently disconnecting from wearable device 100.

In some implementations, back housing 159 is inhibited from rotating in the second direction when first inserted into receptacle 110 (see FIGS. 9B-9C) such that a user can only rotate back housing 159 in the first direction when back housing 159 is first inserted into receptacle 110. In some implementations, a portion of back housing 159 (for example, surface 173) can contact a portion of wearable device 100 (for example, barrier 116) to prevent back housing 159 from rotating in the second direction when back housing 159 is initially inserted into receptacle 110.

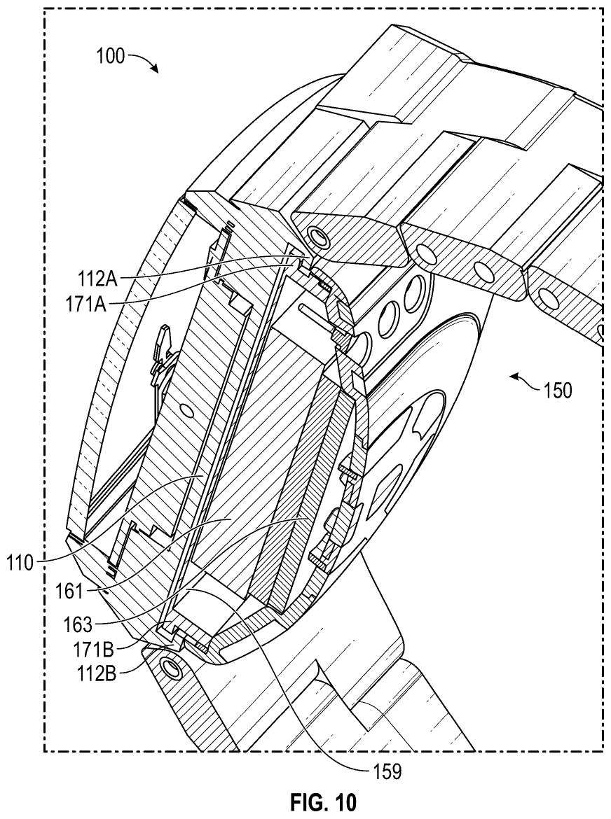

FIG. 10 illustrates a cross-sectional view of wearable device 100 with sensor assembly 150 connected. FIG. 10 illustrates an example of rail 112A and rail 112B mechanically mating with sensor assembly 150 to hold sensor assembly 150 to receptacle 110 and wearable device 100. Rail 112A can be positioned above the bottom surface of receptacle 110 such that there is a recess positioned between rail 112A and the bottom surface of receptacle 110. The recess can be sized and shaped such that rib 171A can slide between rail 112A and receptacle 110 when sensor assembly 150 is rotated in the first direction to secure sensor assembly 150 to wearable device 100. Rail 112A can physically inhibit movement of rib 171A away from wearable device 100 when rib 171A is positioned in the recess such that back housing 159 and/or sensor assembly 150 is held against and connected to receptacle 110 and wearable device 100. Rib 171A can rotate in the first and/or second direction when positioned in the recess such that sensor assembly 150 is also permitted to rotate in the first and/or second direction to connect and/or disconnect to wearable device 100.

Rail 112B can be positioned above the bottom surface of receptacle 110 such that there is a recess positioned between rail 112B and the bottom surface of receptacle 110. The recess can be sized and shaped such that rib 171B can slide between rail 112B and receptacle 110 when sensor assembly 150 is rotated in the first direction to secure sensor assembly 150 to wearable device 100. Rail 112B can physically inhibit movement of rib 171B away from wearable device 100 when rib 171B is positioned in the recess such that the back housing 159 and/or sensor assembly 150 is held against and connected to receptacle 110 and wearable device 100. Rib 171B can rotate in the first and/or second direction when positioned in the recess such that sensor assembly 150 is also permitted to rotate in the first and/or second direction to connect and/or disconnect to wearable device 100.

FIG. 11 illustrates a portion of circuit board 163 of sensor assembly 150. FIG. 12 illustrates a cross-sectional view of a portion of sensor assembly 150. Circuit board 163 can include one or more emitters 179, one or more detectors 177, and/or one or more temperature sensors 181. In some implementations, circuit board 163 can include a plurality of emitters 179 mounted to circuit board 163. In some implementations, the one or more emitters 179 can be mounted on a center portion of circuit board 163. The one or more emitters 179 can emit optical radiation. The one or more emitters 179 can be oriented such that they emit optical radiation through the one or more windows 151 of sensor assembly 150 toward a user's tissue when sensor assembly 150 is connected to wearable device 100 and wearable device 100 is worn by the user.

In some implementations, circuit board 163 can include a plurality of detectors 177 mounted to circuit board 163. In some implementations, a plurality of detectors 177 can be arranged on circuit board 163 to surround the one or more emitters 179. In some implementations, a plurality of detectors 177 can be arranged in a circular array. The one or more detectors 177 can output one or more signals in response to detecting optical radiation emitted by the one or more emitters 179. In some implementations, the one or more detectors 177 can output one or more signals in response to detecting optical radiation emitted by the one or more emitters 179 after attenuation by the tissue of a user. The one or more detectors 177 can be oriented such that they face substantially toward a user's tissue when sensor assembly 150 is connected to wearable device 100 and wearable device 100 is worn by the user.

In some implementations, the one or more temperature sensors 181 can be mounted to circuit board 163. The one or more temperature sensors 181 can respond to thermal energy from a user. In some implementations, the one or more temperature sensors 181 can include thermistors. In some implementations, the one or more temperature sensors 181 can be mounted to a center portion of circuit board 163. In some implementations, the one or more temperature sensors 181 can be surrounded by a plurality of detectors 177.

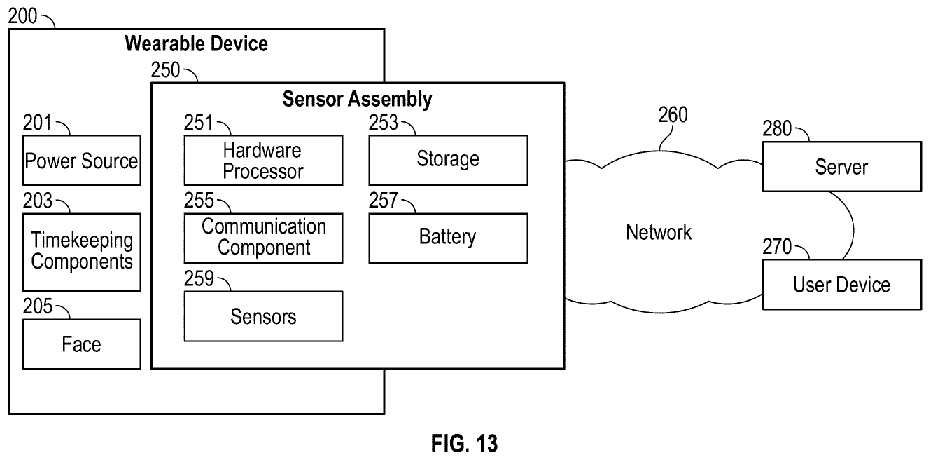

FIG. 13 is a block diagram illustrating a sensor assembly 250 in communication with a server 280 and/or a user device 270 over network 260. The sensor assembly 250 can mechanically integrate with wearable device 200. Sensor assembly 250 can removably connect with wearable device 200 and can operate independent of wearable device 200 whether or not sensor assembly 250 is mechanically connected with wearable device 200. In some implementations, wearable device 200 is a watch, such as a mechanical watch.

Wearable device 200 can include a power source 201 and timekeeping components 203. In some implementations, wearable device 200 does not include electronic components such as an electronic display (for example, an LED/LCD display). Power source 201 may be a mechanical power source. In some implementations, power source 201 is not an electrical power source, such as a battery. Sensor assembly 250 may be electrically isolated from wearable device 200 such that battery 257 does not provide power to wearable device 200. Power source 201 may be a spring configured to store mechanical energy to provide power to the wearable device 200, for example to power the timekeeping components 203.

Timekeeping components 203 can include one or more of a gear train, including one or more gears, a balance wheel, and an escapement. The gear train can transmit power from the power source 201 to the balance wheel. The balance wheel can oscillate to track passage of time. The escapement can provide force to the balance wheel to maintain the balance wheel's oscillation and can also control movement of the gear train by allowing gears to rotate in discrete amounts. Timekeeping components 203 can control face 205 which can display time such as with rotating hands. In some implementations, timekeeping components 203 and/or face 205 operate independently of sensor assembly 250, whether or not sensor assembly is mechanically connected with wearable device 200

Sensor assembly 250 can include one or more hardware processors 251, storage 253, communication component 255, battery 257, and one or more sensors 259. In some implementations, sensor assembly 250 does not have a display.

Hardware processor 251 can comprise one or more integrated circuits. The hardware processor 251 can comprise and/or have access to memory. The hardware processor 251 can comprise and/or be embodied as one or more chips, controllers such as microcontrollers (MCUs), and/or microprocessors (MPUs). The hardware processor 251 can comprise a central processing unit (CPU). In some implementations, the hardware processor 251 can be embodied as a system-on-a-chip (SoC). The hardware processor 251 can be configured to implement an operating system which can allow multiple processes to execute simultaneously. The hardware processor 251 can be configured to execute program instructions to cause the sensor assembly 250 to perform one or more operations. The hardware processor 251 can be configured, among other things, to process data, execute instructions to perform one or more functions, and/or control the operation of the sensor assembly 250 or components thereof. For example, the hardware processor 251 can process physiological data obtained from sensors 259 and can execute instructions to perform functions related to storing and/or transmitting such physiological data. In some implementations, the hardware processor 251 can be remote to the sensor assembly 250.

The hardware processor 251 can access and process photoplethysmography (PPG) data originating from a PPG sensor such as an oximeter. The hardware processor 251 can determine characteristics of pulsatile blood flow from the PPG data. For example, the hardware processor 301 can determine volumetric variations in blood circulation from the PPG data and derive one or more parameters therefrom, such as pulse rate, blood pressure, respiration rate, respiration volume, cardiac output, pleth variability index, PPG waveform data, etc. Hardware processor 251 can also determine other parameters from PPG data such as blood oxygen saturation, hydration, hemoglobin content, perfusion index, etc.

The hardware processor 251 can access and process other physiological data such as temperature data originating from a thermistor and/or infrared sensor to determine body temperature such as core body temperature and/or surface body temperature; and inertial data originating from an inertial sensor such as an accelerometer or gyroscope.

The storage 253 can include any computer readable storage medium and/or device (or collection of data storage mediums and/or devices), including, but not limited to, one or more memory devices that store data, including without limitation, dynamic and/or static random-access memory (RAM), programmable read-only memory (PROM), erasable programmable read-only memory (EPROM), electrically erasable programmable read-only memory (EEPROM), optical disks (e.g., CD-ROM, DVD-ROM, etc.), magnetic disks (e.g., hard disks, floppy disks, etc.), memory circuits (e.g., solid state drives, random-access memory (RAM), etc.), and/or the like. The storage 253 can store data including processed and/or unprocessed physiological data obtained from sensors 259. The storage 253 can store program instructions that when executed by the hardware processor 251 cause the sensor assembly 250 to perform one or more operations.

The communication component 255 can facilitate communication (via wireless, wired, and/or wire-like connection) between the sensor assembly 250 (and/or components thereof) and separate devices, such as server 280 and/or user device 270. For example, the communication component 255 can be configured to allow the sensor assembly 250 to wirelessly communicate with other devices, systems, and/or networks over any of a variety of communication protocols, including near-field communication protocols and far-field communication protocols. Near-field communication protocols, which may also be referred to as non-radiative communication, can implement inductive coupling between coils of wire to transfer energy via magnetic fields (e.g., NFMI). Near-field communication protocols can implement capacitive coupling between conductive electrodes to transfer energy via electric fields. Far-field communication protocols, which may also be referred to as radiative communication, can transfer energy via electromagnetic radiation (e.g., radio waves). The communication component 255 can communicate via any variety of communication protocols such as Wi-Fi, Bluetooth®, ZigBee®, Z-wave®, cellular telephony, such as long-term evolution (LTE) and/or 1G, 2G, 3G, 4G, 5G, etc., infrared, radio frequency identification (RFID), satellite transmission, inductive coupling, capacitive coupling, proprietary protocols, combinations of the same, and the like. In some implementations, communication component 255 can implement human body communication (HBC) which can include capacitively coupling a transmitter and receiver via an electric field propagating through the human body. The communication component 255 can allow data and/or instructions to be transmitted and/or received to and/or from the sensor assembly 250 and separate computing devices. The communication component 255 can be configured to transmit and/or receive (for example, wirelessly) processed and/or unprocessed physiological data with separate computing devices. The communication component 255 can be embodied in one or more components that are in communication with each other. The communication component 255 can include one or more of: transceivers, antennas, transponders, radios, emitters, detectors, coils of wire (e.g., for inductive coupling), and/or electrodes (e.g., for capacitive coupling). The communication component 255 can include one or more integrated circuits, chips, controllers, processors, or the like, such as a Wi-Fi chip and/or a Bluetooth chip.

Battery 257 can provide power for components of the sensor assembly 250. Battery 257 can connect to an external power source to receive power from the external power source. Battery 257 can include a dual-battery configuration with a main battery and a backup battery. Battery 257 can include a lithium based battery such as a lithium ion battery and/or a lithium polymer battery.

The sensors 259 can include one or more physiological sensors configured to generate physiological data of physiological parameters. The sensors 259 can include acoustic sensors, optical sensors, inertial sensors (such as an accelerometer and/or gyroscope), temperature sensors, ECG electrodes, etc. The sensors 259 can include an oximeter configured to generate photoplethysmography (PPG) data useable to determine physiological information such as volumetric changes in pulsatile blood flow, blood oxygen saturation, hydration, hemoglobin content, pulse rate, blood pressure, respiration rate, respiration volume, cardiac output, perfusion index, pleth variability index, PPG waveform data, etc. The sensors 259 can include one or more optical emitters configured to emit optical radiation of a plurality of wavelengths, which can include visible light, near-infrared, and/or infrared. The sensors 259 can include one or more optical detectors configured to detect optical radiation attenuated by the tissue of a subject (which may have been emitted by optical emitters) and generate PPG data usable to determine pulsatile characteristics of the subject such as blood oxygen saturation, etc. The sensors 259 can measure and/or generate data relating to respiration rate, blood oxygen saturation (e.g., SpO2), heart rate, pulse rate, skin temperature, core body temperature, spatial orientation, or the like.

The network 260 can include one or more communications networks. The network 260 can include a plurality of computing devices configured to communicate with one another. The network 260 can include routers. The network 260 can include the Internet. The network 260 can include a cellular network. The network 260 can include any combination of a body area network (e.g., implementing human body communication with capacitive coupling via the tissue of a user's body), a local area network (“LAN”) and/or a wide area network (“WAN”), or the like. Accordingly, various computing devices can communicate with one another directly or indirectly via any appropriate communications links and/or networks, such as network 260 (e.g., one or more communications links, one or more computer networks, one or more wired or wireless connections, the Internet, any combination of the foregoing, and/or the like).

Communication over the network 260 can include a variety of communication protocols, including wired communication, wireless communication, wire-like communication, near-field communication (such as inductive coupling between coils of wire or capacitive coupling between conductive electrodes), and far-field communication (such as transferring energy via electromagnetic radiation (e.g., radio waves)). Example communication protocols can include Wi-Fi, Bluetooth®, ZigBee®, Z-wave®, cellular telephony, such as long-term evolution (LTE) and/or 1G, 2G, 3G, 4G, 5G, etc., infrared, radio frequency identification (RFID), satellite transmission, inductive coupling, capacitive coupling, proprietary protocols, combinations of the same, and the like.

The server 280 can comprise one or more computing devices including one or more hardware processors. The server 280 can comprise program instructions configured to cause the server 280 to perform one or more operations when executed by the hardware processors. The server 280 can include, and/or have access to (e.g., be in communication with and/or host) a storage device, database, or system which can include any computer readable storage medium and/or device (or collection of data storage mediums and/or devices), including, but not limited to, one or more memory devices that store data, including without limitation, dynamic and/or static random-access memory (RAM), programmable read-only memory (PROM), erasable programmable read-only memory (EPROM), electrically erasable programmable read-only memory (EEPROM), optical disks (e.g., CD-ROM, DVD-ROM, etc.), magnetic disks (e.g., hard disks, floppy disks, etc.), memory circuits (e.g., solid state drives, random-access memory (RAM), etc.), and/or the like. In some implementations, the server 280 can include and/or be in communication with a hosted storage environment that includes a collection of physical data storage devices that may be remotely accessible and may be rapidly provisioned as needed (commonly referred to as “cloud” storage). Data stored in and/or accessible by the server 280 can include physiological data including physiological data previously obtained by the one or more sensors 259 and/or processed by hardware processor 251. In some implementations, the server 280 can comprise and/or be in communication with an electronic medical records (EMR). An EMR can comprise a propriety EMR. An EMR can comprise an EMR associated with a hospital. An EMR can store data including medical records.

User device 270 may be a computing device and can include one or more hardware processors capable of executing program instructions. User device 270 may be a smartphone, laptop, tablet, computer, monitoring hub, sensors, home automation system, exercise device, vehicle, auricular device, audio device, or the like. User device 270 may be an electronic wearable device and may be worn at the same time as wearable device 200.

### Additional Considerations

Certain categories of persons, such as caregivers, clinicians, doctors, nurses, and friends and family of a user, may be used interchangeably to describe a person providing care to the user. Furthermore, patients or users used herein interchangeably refer to a person who is wearing a sensor or is connected to a sensor or whose measurements are used to determine a physiological parameter or a condition. Parameters may be, be associated with, and/or be represented by, measured values, display icons, alphanumeric characters, graphs, gages, power bars, trends, or combinations. Real time data may correspond to active monitoring of a user, however, such real time data may not be synchronous to an actual physiological state at a particular moment. Measurement value(s) of a parameter and the parameter used herein such as, SpO2, RR, PaO2 and the like, unless specifically stated otherwise, or otherwise understood with the context as used is generally intended to convey a measurement or determination that is responsive to the physiological parameter.

Although certain implementations and examples have been described herein, it will be understood by those skilled in the art that many aspects of the systems and devices shown and described in the present disclosure may be differently combined and/or modified to form still further implementations or acceptable examples. All such modifications and variations are intended to be included herein within the scope of this disclosure. A wide variety of designs and approaches are possible. No feature, structure, or step disclosed herein is essential or indispensable. The various features and processes described herein may be used independently of one another, or may be combined in various ways. For example, elements may be added to, removed from, or rearranged compared to the disclosed example implementations. All possible combinations and sub-combinations are intended to fall within the scope of this disclosure.

Any methods and processes described herein are not limited to any particular sequence, and the blocks or states relating thereto can be performed in other sequences that are appropriate. For example, described blocks or states may be performed in an order other than that specifically disclosed, or multiple blocks or states may be combined in a single block or state, or certain method or process blocks may be omitted, or certain blocks or states may be performed in a reverse order from what is shown and/or described. The example blocks or states may be performed in serial, in parallel, or in some other manner. Blocks or states may be added to or removed from the disclosed example implementations.

The methods disclosed herein may include certain actions taken by a practitioner; however, they can also include any third-party instruction of those actions, either expressly or by implication.

The methods and tasks described herein may be performed and fully automated by a computer system. The computer system may, in some cases, include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (e.g., solid state storage devices, disk drives, etc.). The various functions disclosed herein may be embodied in such program instructions, and/or may be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid state memory chips and/or magnetic disks, into a different state. The computer system may be a cloud-based computing system whose processing resources are shared by multiple distinct entities or other users. The systems and modules may also be transmitted as generated data signals (for example, as part of a carrier wave or other analog or digital propagated signal) on a variety of computer-readable transmission mediums, including wireless-based and wired/cable-based mediums, and may take a variety of forms (for example, as part of a single or multiplexed analog signal, or as multiple discrete digital packets or frames).

Many other variations than those described herein will be apparent from this disclosure. For example, depending on the implementation, certain acts, events, or functions of any of the algorithms described herein can be performed in a different sequence, can be added, merged, or left out altogether (for example, not all described acts or events are necessary for the practice of the algorithms). Moreover, in certain implementations, acts or events can be performed concurrently, for example, through multi-threaded processing, interrupt processing, or multiple processors or processor cores or on other parallel architectures, rather than sequentially. In addition, different tasks or processes can be performed by different machines and/or computing systems that can function together.

Various illustrative logical blocks, modules, routines, and algorithm steps that may be described in connection with the disclosure herein can be implemented as electronic hardware (e.g., ASICs or FPGA devices), computer software that runs on computer hardware, or combinations of both. Various illustrative components, blocks, and steps may be described herein generally in terms of their functionality. Whether such functionality is implemented as specialized hardware versus software running on general-purpose hardware depends upon the particular application and design constraints imposed on the overall system. The described functionality can be implemented in varying ways for each particular application, but such implementation decisions should not be interpreted as causing a departure from the scope of the disclosure.

Moreover, various illustrative logical blocks and modules that may be described in connection with the implementations disclosed herein can be implemented or performed by a machine, such as a general purpose processor, a digital signal processor (“DSP”), an application specific integrated circuit (“ASIC”), a field programmable gate array (“FPGA”) or other programmable logic device, discrete gate or transistor logic, discrete hardware components, or any combination thereof designed to perform the functions described herein. A general purpose processor can be a microprocessor, controller, microcontroller, or state machine, combinations of the same, or the like. A processor can include electrical circuitry configured to process computer-executable instructions. A processor can include an FPGA or other programmable devices that performs logic operations without processing computer-executable instructions. A processor can also be implemented as a combination of computing devices, for example, a combination of a DSP and a microprocessor, a plurality of microprocessors, one or more microprocessors in conjunction with a DSP core, or any other such configuration. Although described herein primarily with respect to digital technology, a processor may also include primarily analog components. For example, some, or all, of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of any method, process, routine, or algorithm described in connection with the disclosure herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC. The ASIC can reside in a user terminal. In the alternative, the processor and the storage medium can reside as discrete components in a user terminal.

Conditional language used herein, such as, among others, “can,” “could,” “might,” “may,” “e.g.,” and the like, unless specifically stated otherwise, or otherwise understood within the context as used, is generally intended to convey that certain features, elements, and/or steps are optional. Thus, such conditional language is not generally intended to imply that features, elements, and/or steps are in any way required or that one or more embodiments necessarily include logic for deciding, with or without other input or prompting, whether these features, elements, and/or steps are included or are to be always performed. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 10 degrees, 5 degrees, 3 degrees, or 1 degree. As another example, in certain embodiments, the terms “generally perpendicular” and “substantially perpendicular” refer to a value, amount, or characteristic that departs from exactly perpendicular by less than or equal to 10 degrees, 5 degrees, 3 degrees, or 1 degree.

As used herein, “real-time” or “substantial real-time” may refer to events (e.g., receiving, processing, transmitting, displaying etc.) that occur at a same time as each other, during a same time as each other, or overlap in time with each other. “Real-time” may refer to events that occur at distinct or non-overlapping times the difference between which is imperceptible and/or inconsequential to humans such as delays arising from electrical conduction or transmission. A human may perceive real-time events as occurring simultaneously, regardless of whether the real-time events occur at an exact same time. As a non-limiting example, “real-time” may refer to events that occur within a time frame of each other that is on the order of milliseconds, seconds, tens of seconds, or minutes. For example, “real-time” may refer to events that occur within a time frame of less than 1 minute, less than 30 seconds, less than 10 seconds, less than 1 second, less than 0.05 seconds, less than 0.01 seconds, less than 0.005 seconds, less than 0.001 seconds, etc.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

As used herein, “system,” “instrument,” “apparatus,” and “device” generally encompass both the hardware (for example, mechanical and electronic) and, in some implementations, associated software (for example, specialized computer programs for operational control) components.

It should be emphasized that many variations and modifications may be made to the herein-described implementations, the elements of which are to be understood as being among other acceptable examples. All such modifications and variations are intended to be included herein within the scope of this disclosure. Any section headings used herein are merely provided to enhance readability and are not intended to limit the scope of the implementations disclosed in a particular section to the features or elements disclosed in that section. The foregoing description details certain implementations. It will be appreciated, however, that no matter how detailed the foregoing appears in text, the systems and methods can be practiced in many ways. As is also stated herein, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated.

Those of skill in the art would understand that information, messages, and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

While the above detailed description has shown, described, and pointed out novel features, it can be understood that various omissions, substitutions, and changes in the form and details of the devices or algorithms illustrated can be made without departing from the spirit of the disclosure. As can be recognized, certain portions of the description herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

What is claimed is:

1. A sensor assembly, comprising:
a housing comprising one or more ribs configured to slide within a mechanically powered watch to removably mechanically connect the sensor assembly with the mechanically powered watch, said housing having a surface configured to contact tissue of a user when the sensor assembly is connected with the mechanically powered watch and the mechanically powered watch is worn by the user;
a circuit board housed by the housing;
a plurality of emitters mounted to the circuit board and configured to emit optical radiation through one or more windows of the housing toward tissue of the user;
a plurality of detectors mounted to the circuit board, said plurality of detectors configured to output one or more signals responsive to detecting optical radiation emitted from the plurality of emitters after attenuation by said tissue;
one or more hardware processors configured to process the one or more signals from the plurality of detectors to generate processed physiological data;
a communication component configured to communicate the processed physiological data to a remote computing device; and
one or more batteries housed by the housing and configured to provide power to the circuit board, the one or more hardware processors, and the communication component; and
wherein the sensor assembly is not electrically connected with the mechanically powered watch and is configured to operate independently of the mechanically powered watch.

2. The sensor assembly of claim 1, further comprising one or more charging ports embedded within the surface and housing one or more charging pins, wherein said one or more charging pins are electrically connected with the one or more batteries.

3. The sensor assembly of claim 1, wherein the sensor assembly is electrically isolated from the mechanically powered watch.

4. The sensor assembly of claim 1, further comprising a shroud removably coupled with the housing.

5. The sensor assembly of claim 4, wherein the shroud forms at least a portion of the surface when coupled with the housing.

6. The sensor assembly of claim 5, wherein the shroud surrounds the one or more windows.

7. The sensor assembly of claim 5, wherein the shroud has a different color and/or texture than the housing.

8. The sensor assembly of claim 1, further comprising a temperature sensor mounted to the circuit board and configured to respond to thermal energy from the user.

9. The sensor assembly of claim 1, wherein the housing is configured to:
rotate in a first direction relative to the mechanically powered watch to mechanically connect the housing to the mechanically powered watch; and
rotate in a second direction relative to the mechanically powered watch to mechanically disconnect the housing from the mechanically powered watch.

10. The sensor assembly of claim 9, wherein the housing comprises a surface configured to contact a wall of the mechanically powered watch to inhibit the sensor assembly from over rotating in the first direction.

11. The sensor assembly of claim 1, wherein at least a portion of the sensor assembly is circular.

12. The sensor assembly of claim 1, wherein the surface of the housing is convex.

13. A mechanically powered watch, comprising:
a face disposed on a front portion of the mechanically powered watch, said face configured to indicate time;
timekeeping components comprising one or more gears configured to control the time indicated by the face;
a spring configured to provide power to the timekeeping components; and
a receptacle on a back portion of the mechanically powered watch opposite the front portion, the receptacle configured to removably mechanically connect with an electronic sensor assembly, the receptacle configured to hold the electronic sensor assembly against tissue of a user when the mechanically powered watch is worn by the user.

14. The mechanically powered watch of claim 13, further comprising a rail configured to mechanically mate with the sensor assembly to hold the sensor assembly to the receptacle.

15. The mechanically powered watch of claim 13, wherein the mechanically powered watch is configured to:
rotate in a first direction relative to the sensor assembly to mechanically connect the sensor assembly with the receptacle; and
rotate in a second direction relative to the sensor assembly to mechanically disconnect the sensor assembly from the receptacle.

16. The mechanically powered watch of claim 15, further comprising a barrier configured to inhibit the sensor assembly from rotating in the first direction beyond a desired orientation relative to the receptacle.

17. The mechanically powered watch of claim 13, wherein the receptacle is configured to removably mechanically connect with a cover when the electronic sensor assembly is mechanically disconnected from the receptacle.

18. The mechanically powered watch of claim 13, wherein the timekeeping components operate independently from the electronic sensor assembly and continue to operate when the electronic sensor assembly is mechanically disconnected from the receptacle.

19. The mechanically powered watch of claim 13, wherein the mechanically powered watch does not have an electronic display.

20. The mechanically powered watch of claim 13, wherein the mechanically powered watch does not have a battery.
